(19) United States
(12) Patent Application Publication
(10) Pub. No.: US 2024/0216469 A1
(43) Pub. Date: Jul. 4, 2024

(54) SYNTHETIC TISSUE BARRIERS AND USES THEREOF

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Ameya R. Kirtane, Arlington, MA (US); Junwei Li, Cambridge, MA (US); Thomas Wang, Chicago, IL (US); James Byrne, Boston, MA (US); Alexis Jones, Cambridge, MA (US)

(73) Assignees: Massachusetts Institute of Technology, Cambridge, MA (US); The Brigham and Women's Hospital, Inc., Boston, MA (US)

(21) Appl. No.: 18/596,376

(22) Filed: Mar. 5, 2024

Related U.S. Application Data
(63) Continuation of application No. 17/784,598, filed on Jun. 10, 2022.
(60) Provisional application No. 63/050,206, filed on Jul. 10, 2020.
(60) Provisional application No. 63/050,216, filed on Jul. 10, 2020.
(60) Provisional application No. 62/947,582, filed on Dec. 13, 2019.

(51) Int. Cl. A61K 38/17; A61K 31/495; A61K 45/06; A61L 24/04; A61L 24/06; A61L 24/08; A61L 27/16; A61L 27/18; A61L 27/20; C12N 9/08

(52) U.S. Cl. CPC A61K 38/1787; A61K 31/495; A61K 45/06; A61L 24/046; A61L 24/06; A61L 24/08; A61L 27/16; A61L 27/18; A61L 27/20; C12N 9/0065; A61L 2300/254; A61L 2300/256; A61L 2300/438; A61L 2400/04; A61L 2430/06; A61L 2430/16; C12Y 111/01006

(57) ABSTRACT

The present disclosure provides compositions, methods, and kits that enable the in situ growth of polymers on or within a subject. In some aspects, the tissue-active monomers, including monomers comprising macromolecules, provide a broad set of material choices for synthetic tissue barriers. In additional aspects, the compositions, methods, and kits are useful for treating or preventing a disease or disorder.

### CROSS-REFERENCE TO RELATED APPLICATIONS

This application claims priority under 35 U.S.C. § 119(e) to U.S. Provisional Patent Application, U.S. Ser. No. 62/947,582, filed Dec. 13, 2019, U.S. Provisional Patent Application, U.S. Ser. No. 63/050,206, filed Jul. 10, 2020, and U.S. Provisional Patent Application, U.S. Ser. No. 63/050,216, filed Jul. 10, 2020, the contents of each of which are incorporated herein by reference in their entireties.

### BACKGROUND OF THE INVENTION

Tissue barriers covering most parts of the body are not only protective shields against physical abrasion, chemical stress, environmental stress, xenobiotics, and microbial pathogens, but also dynamic linings for signal sensing (e.g., of temperature, viruses, and bacteria), molecule transport (e.g., of oxygen, nutrients, and drugs), and immunity coordination (e.g., cytokine, mucin, and host-defense peptides).⁹⁻¹⁹ Disruption of tissue barrier functions is associated with the pathogenesis of many human diseases.¹⁴⁻¹⁹ Selective intervention of a specific tissue barrier is pertinent in disease treatment and health management. In parallel, to address the challenge of efficiently and specifically restoring or augmenting functions of tissue barriers, a large variety of meticulously designed biotechnologies have been developed. These particular technologies, leveraging either tissue adhesives or tissue substitutes, are effective tools for facilitating restoration of tissue barrier dysfunctions and treatment of systemic diseases^{[0001]1,[0001]2,[0001]3,[0001]4[0001]4,[0001]5,[0001]6}. Despite these advances in research laboratories, broad adoption of these technologies in medical laboratories and healthcare clinics has been limited, consequently stifling their impact. This narrow implementation is a result of multiple factors: invasive transplantation, potential immunogenicity, toxicity, and most importantly the inaccuracy, instability and inconvenience of current tissue targeting strategies, restricting selective tissue barrier access.

### SUMMARY OF THE INVENTION

Described herein are compositions, methods, and kits that enable the growth of monomers such as polydopamine (PDA) in contact with epithelial tissue and provide a nontoxic, stable, transient synthetic tissue barrier with adjustable functions, which allows for easy, non-invasive transplantation. The polymeric coating relies on dopamine polymerization catalyzed by an endogenous cellular enzyme (e.g., catalase or “CAT”), strong tissue-adhesion generated through chemical crosslinking, and, optionally, functional agents incorporated through facile conjugation. The present disclosure relates in part to a library of tissue-active monomers, including monomers comprising macromolecules, that provides a broad set of material choices for synthetic tissue barriers. Based on a whole-body survey, the tissue-accelerated polymerization material library allows for the provision of new formulations applied in different tissues sites for various therapeutic strategies, including for the treatment of hemostasis (e.g., in upper gastrointestinal bleeding), and improvement of administration efficiency (e.g., sustained drug release) in the eye conjunctiva, cartilage, and oral cavity (e.g., protection of epithelium against environmental stresses in the oral cavity).

In one aspect, provided herein is a method of forming a polymer in situ in a subject, the method comprising administering to the subject a composition comprising a monomer and an oxygen source, wherein the monomer and oxygen source contact a catalyst endogenous to the subject in vivo, and wherein the catalyst polymerizes the monomer.

In one aspect, the disclosure provides a composition comprising (i) a monomer selected from dopamine, levodopa, norepinephrine, methyldopa, levodopa methyl ester, levodopa ethyl ester, or a monomer comprising: dopamine, levodopa, norepinephrine, methyldopa, levodopa methyl ester, or levodopa ethyl ester and a macromolecule selected from one or more of alginate, hyaluronic acid, polyacrylic acid, polyethylene glycol, chondroitin sulfate, or chitosan, (ii) an oxygen source, (iii) optionally, a buffer, and (iv) optionally, an agent.

In another aspect, the disclosure provides a method of treating a disease or disorder comprising administering an effective amount of a composition as described herein to a subject in need thereof.

Further provided are methods of preventing a disease or disorder comprising administering an effective amount of a composition as described herein to a subject in need thereof.

In a further aspect, provided herein is a method of treating bleeding, comprising administering an effective amount of a composition as described herein to a subject in need thereof.

The disclosure also provides kits comprising a composition as described herein and instructions for administering the same.

The details of certain embodiments of the invention are set forth in the Detailed Description of Certain Embodiments, as described below. Other features, objects, and advantages of the invention will be apparent from the Definitions. Examples, and Claims.

### BRIEF DESCRIPTION OF THE DRAWINGS

FIGS. 1A-1E show Tissue-accelerated polymerization technology and whole-body screening of tissue catalytic activities. FIG. 1A is a schematic illustration of the synthetic tissue barrier technology. Tissue-accelerated polymerization monomers are rapidly oxidized under endogenous catalase catalysis, and FIG. 1B is a formed polymeric tissue-accelerated polymerization coating on tissues. FIG. 1C is a schematic illustration of tissue-accelerated polymerization active tissues in the body, where tissues are divided by main body systems, including respiratory, urinary, lymphatic, circulatory, integumentary system, skeletal, muscular, nervous, digestive and endocrine systems. Tissue-accelerated polymerization positive (active) sites are labelled in dark-brown color, which includes the trachea, lung, kidney, bladder, lymph node, heart, blood vessel, skin, cartilage, muscle, nerve, mouth, liver, spleen, gallbladder, pancreas, and small bowel. FIG. 1D shows images showing tissue samples in different parts of the tissues before and after the tissue-accelerated polymerization process. Samples (6 mm in diameter) were collected at three random sites of polydopamine coated tissue. FIG. 1E shows quantitative measurements of the polydopamine signal intensities of samples shown in FIG. 1D.

FIGS. 2A-2C show a library of tissue-accelerated polymerization monomers wherein some monomers comprise macromolecules. FIG. 2A shows tissue-accelerated polymerization monomers. FIG. 2B shows schematic illustration of tissue-accelerated polymerization molecule library that includes 6 tissue-accelerated polymerization monomers and 21 tissue-accelerated polymerization macromolecules. FIG. 2C shows a heat map showing increase of polymerization rates under catalase catalysis for 1,142 separate monomer formulations.

FIG. 3 shows an image of the assembly of tissue-directed polymer assembling (“TPA”) monomers M1-M6 with and without addition of CAT. The top row shows that TPA monomers M1-M6 do not initially polymerize without the addition of CAT. With the addition of CAT in the bottom row, assembly of TPA monomers M1-M6 is observed.

FIGS. 4A-4F show normalized extinction spectra of polymeric tissue-accelerated polymerization standard and polymerization products. (FIGS. 4A-4F, M1-M6, respectively) UV-Vis spectra of polymeric tissue-accelerated polymerization standard (dot line) and polymerization product (solid line) under catalase are nearly identical, confirming the polymeric tissue-accelerated polymerization formation under catalase catalysis.

FIGS. 5A-5F show TPA assembly kinetics. Optical extinction of TPA monomer assembly solutions was measured at 500 nm for solutions containing 6 monomers (FIGS. 5A-5F, M1-M6) with or without catalase catalysis over 3 hours. In the presence of CAT, light extinction intensities rapidly grew and plateaued within several minutes of reaction initialization. In the absence of CAT, signals were minimal even after 3 hours, of which the same intensity was reached within 1-2 minutes under CAT-catalyzed conditions, demonstrating an acceleration of assembly rate by about 100-200 times for all TPA monomers.

FIG. 6 shows a photograph of TPA monomer coating on porcine cartilage. An image of ex vivo tissue samples of porcine cartilage after incubation with each TPA monomer solution and washing to remove excess unbound TPA assemblies in the solution is shown. Distinct color change is present in all tissue samples after incubation as compared to the control.

FIGS. 7A-7C show TPA-macromolecule conjugate illustrations. Schematic illustrations of 21 different TPA-macromolecule conjugates formed through the combination of TPA monomers with six different carboxyl-rich macromolecules (sodium alginate, hyaluronate, polyacrylic acid, polyethylene glycol, chondroitin sulfate and chitosan) are shown.

FIGS. 8A-8C show NMR spectra of 21 tissue-accelerated polymerization monomers comprising macromolecules. Unconjugated macromolecules (marked with an asterisk (*)), tissue-accelerated polymerization monomers (marked with an arrow ( [symbol] ), and conjugated tissue-accelerated polymerization macromolecules (unmarked) are shown.

FIG. 9 shows heat maps illustrating TPA assembly biocompatibility. Cell viability of Caco-2 and HeLa cells is shown upon administration of TPA assemblies (formed from the combination of M1-M6 monomers and C1-C21 TPA-macromolecule conjugates) and 24-hour in vitro incubation. Polymer assemblies generated from C7-C12 show relatively lower cytotoxicity.

FIG. 10 shows TPA eye drop application with conjunctiva specificity. A schematic illustration of TPA eye drop application and specific assembly on the conjunctiva of the human eye is shown. A brown color is observed after TPA eye drops, an optical characteristic of TPA polymerization and adhesion.

FIGS. 11A-11O show TPA for drug delivery, tissue protection, and hemostasis treatment.

FIG. 12 shows TPA assembly adhesion to the conjunctiva. Images of porcine eye and conjunctiva after TPA eye drop application before and after rinsing with an isotonic solution are shown. TPA assembly remains intact after rinsing.

FIG. 13 shows transient coating of TPA assembly on the conjunctiva. An image of a porcine eye and conjunctiva 24 hours after TPA eye drop application is shown. The conjunctiva is pink and free of TPA assembly.

FIG. 14 shows histology of porcine conjunctiva 24 hours after TPA administration compared to control.

FIG. 15 shows stability of the TPA patch after vigorous washing. Images of porcine tongue after application and adhesion of TPA patch before and after vigorous rinsing are shown. The TPA patch is dark brown, an optical characteristic of polymerization and adhesion of the TPA assembly layer, and remains so after rinsing.

FIG. 16 shows transient adhesion of TPA patch after 24 hours. An image of a porcine tongue 24 hours after adhesion of a TPA patch where the patch and TPA assembly is no longer adhered to or present on the tissue surface is shown.

FIG. 17 shows histology of porcine tongue epithelium after TPA patch adhesion.

FIG. 18 shows stability of the TPA-lead patch over 3 hours. X-ray images of the porcine head profile after administration of TPA-lead patches onto the buccal surface of sedated porcine are shown. The TPA-lead patch is fixed in its original orientation after 3 hours.

FIG. 19 shows blood gelation upon mixture with TPA powder. Images are shown of fresh porcine blood collected through tubes containing sodium citrate anti-coagulant, and then mixed with the TPA hemostatic powder (M1 and C7 as TPA molecules). The blood (red liquid) instantly turned to a black gel once mixed with the TPA powder.

FIGS. 20A-20B show images of in vivo hemostasis studies. Endoscopy images showing the bleeding site before and after tissue-accelerated polymerization powder treatment, as in FIG. 20A, and tissue-accelerated polymerization powder without H₂O₂ as control treatment, as in FIG. 20B. Images from gastrointestinal endoscopy videography of acute stomach-bleeding pig model with endoscopically applied powder without the TPA gelation ingredients (control) are shown. Under moderate sedation, endoscopic biopsy forceps were utilized to generate a 10-20 mm puncture wound (without perforation) in the stomach, resulting in bleeding (oozing hemorrhage) from the puncture site. Ten minutes after the endoscopic powder application, no color change or reduction in bleeding is present.

FIG. 21 shows images of tissue recovery after hemostasis treatment. Endoscopy images showing bleeding sites in the stomach after 24 hours. Different treatments were performed on the bleeding site. Tissue-accelerated polymerization powder can help tissue regeneration and recovery with non-obvious scar and smooth tissue surface. From left to right are shown: 1) an image of porcine stomach after puncture and subsequent TPA hemostasis treatment where a flat wound is observed at the puncture site; 2) an image of porcine stomach 24 hours after puncture and application of control powder, and 3) an image of porcine stomach 24 hours after puncture with no subsequent treatment.

FIG. 22 shows histology of damaged tissues after hemostasis treatment. Histology images showing damaged/bleeding tissues in the stomach after 24 hours. Different treatments were performed on the bleeding site. Tissue-accelerated polymerization powder can help the tissue regeneration and recovery, where tissues recover to similar status with healthy tissues.

FIGS. 23A-23F show the evaluation of TPA in human tissues. FIG. 23A shows that M1 assemblies stably adhere across various human tissues. FIG. 23B shows quantitative signal analysis to confirm TPA barriers on tissue surfaces. FIGS. 23C-23F show M1 assembly formation kinetics.

### DEFINITIONS

Unless otherwise required by context, singular terms shall include pluralities, and plural terms shall include the singular.

The language “in some embodiments” and “in certain embodiments” are used interchangeably.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

When a range of values (“range”) is listed, it is intended to encompass each value and sub-range within the range. A range is inclusive of the values at the two ends of the range unless otherwise provided.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a composition described herein in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a polymer or composition described herein refers to an amount sufficient to elicit the desired biological response. An effective amount of a polymer or composition described herein may vary depending on such factors as the desired biological endpoint, the pharmacokinetics of the polymer or composition, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound, polymer, or composition described herein in a single dose. In certain embodiments, an effective amount is the combined amounts of a compound, polymer, or composition described herein in multiple doses.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes.

The term “polymer” refers to a compound comprising eleven or more covalently connected repeating units. In certain embodiments, a polymer is naturally occurring. In certain embodiments, a polymer is synthetic (i.e., not naturally occurring).

As used herein, the term “assembly” or “assembling” refers to the formation of a polymer by covalent connection of repeating units. For example, a polymer may be assembled from any of the monomers disclosed herein.

The term “nanoparticle” refers to a particle having an average (e.g., mean) dimension (e.g., diameter) of between about 1 nanometer (nm) and about 1 micrometer (μm) (e.g., between about 1 nm and about 300 nm, between about 1 nm and about 100 nm, between about 1 nm and about 30 nm, between about 1 nm and about 10 nm, or between about 1 nm and about 3 nm), inclusive.

As used herein, the term “agent” means a molecule, group of molecules, complex or substance administered to an organism for diagnostic, therapeutic, preventative medical, or veterinary purposes. In certain embodiments, the agent is an active pharmaceutical agent, a diagnostic agent, or a prophylactic agent). In certain embodiments, the polymers and compositions disclosed herein comprise an agent(s), e.g., a first agent (e.g., at least one (including, e.g., at least two, at least three). In some embodiments, the polymers and compositions can further comprise a second agent. In some embodiments, the agent is an enzyme (e.g., a digestive enzyme), a nutrient blocker (e.g., a crosslinking agent), an aptamer, an antibody, a neutralizing agent, a diagnostic agent, a radioprotective agent, a nutraceutical, an active pharmaceutical agent, or a combination thereof.

As used herein, the term “radioprotective agent” means an agent that protects biological systems exposed to radiation, either naturally or through radiation leakage. In certain embodiments, radioprotective agents protect normal cells from radiation injury in cancer patients undergoing radiotherapy

As used herein, the term “neutralizing agent” means an agent that neutralizes an acid or base. In some embodiments, the neutralizing agent is an acid. In some embodiments, the neutralizing agent is a base. In certain embodiments, the neutralizing agent is a buffer.

As used herein, the term “diagnostic agent” means an imaging agent or contrast agent. The terms “imaging agent” and “contrast agent” refer to a substance used to enhance the contrast of structures or fluids within the body in medical imaging. It is commonly used to enhance the visibility of blood vessels and the gastrointestinal tract in medical imaging.

As used herein, the term “active pharmaceutical agent” includes an agent that is capable of providing a local or systemic biological, physiological, or therapeutic effect in the biological system to which it is applied. For example, an active pharmaceutical agent can act to control tumor growth, control infection or inflammation, act as an analgesic, promote anti-cell attachment, and enhance bone growth, among other functions. Other suitable active pharmaceutical agents can include anti-viral agents, hormones, antibodies, or therapeutic proteins. Other active pharmaceutical agents include prodrugs, which are agents that are not biologically active when administered but, upon administration to a subject are converted to biologically active agents through metabolism or some other mechanism.

An active pharmaceutical agent can be a compound, e.g., small organic or inorganic molecules; saccharines; oligosaccharides; polysaccharides; biological macromolecule, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof.

Examples of active pharmaceutical agents include, but are not limited to, antimicrobial agents, analgesics, antinflammatory agents, counterirritants, coagulation modifying agents, diuretics, sympathomimetics, anorexics, antacids and other gastrointestinal agents; antiparasitics, antidepressants, anti-hypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anti-cancer properties, or a combination thereof. Other suitable active pharmaceutical agents include contraceptives and vitamins as well as micro- and macronutrients. Still other examples include antiinfectives such as antibiotics and antiviral agents; analgesics and analgesic combinations: anorexics; antiheimintics; antiarthritics; antiasthmatic agents; anticonvulsants; antidepressants; antidiuretic agents; antidiarrleals; antihistamines; antiinflammatory agents; antimigraine preparations; antinauseants; antineoplastics; antiparkinsonism drugs; antipruritics; antipsychotics; antipyretics, antispasmodics; anticholinergics: sympathomimetics; xanthine derivatives; cardiovascular preparations including calcium channel blockers and beta-blockers such as pindolol and antiarrhythmics; anti-hypertensives; diuretics; vasodilators including general coronary, peripheral and cerebral; central nervous system stimulants; cough and cold preparations, including decongestants: hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins. The disclosure is not intended to be limited in any manner by the above exemplary terms. Additional terms may be defined in other sections of this disclosure.

### DETAILED DESCRIPTION OF CERTAIN EMBODIMENTS

Before the disclosed systems, hydrogels compositions, methods, uses, and kits are described in more detail, it should be understood that the aspects described herein are not limited to specific embodiments, methods, systems, apparati, or configurations, and as such can, of course, vary. It is also to be understood that the terminology used herein is for the purpose of describing particular aspects only and, unless specifically defined herein, is not intended to be limiting.

### Methods and Uses

In one aspect, the disclosure provides a method of forming a polymer in situ in a subject, the method comprising administering to a subject a composition comprising a monomer and an oxygen source, wherein the monomer and the oxygen source contact a catalyst endogenous to the subject and the catalyst polymerizes the monomer.

In some embodiments, the monomer is catalyzed when the oxygen source and monomer contact the endogenous catalyst. In some embodiments, the monomer is catalyzed when the oxygen source and monomer contact a blood cell.

In some embodiments, the oxygen source is hydrogen peroxide or urea hydrogen peroxide. In some embodiments, the oxygen source is hydrogen peroxide. In some embodiments, the oxygen source is urea hydrogen peroxide.

In some embodiments, the endogenous catalyst is selected from catalases or peroxidases. In some embodiments, the endogenous catalyst is a peroxidase. In certain embodiments, the peroxidase is eosinophil peroxidase, lactoperoxidase, or myeloperoxidase.

In some embodiments, the endogenous catalyst is a catalase. In some embodiments, the catalase is a bacterial catalase. In some embodiments, the catalase is a human catalase.

In certain embodiments, the endogenous catalyst is located in the respiratory, urinary, lymphatic, circulatory, integumentary system, skeletal, muscular, nervous, digestive, or endocrine system. In some embodiments, the endogenous catalyst is located in the gastrointestinal (GI) tract of the subject. In some embodiments, the endogenous catalyst is located in the upper GI of the subject. In some embodiments, the endogenous catalyst is located in the gut of the subject. In some embodiments, the endogenous catalyst is located in the stomach of the subject. In some embodiments, the endogenous catalyst is located in a cell. In some embodiments, the endogenous catalyst is located in a blood cell. In some embodiments, the endogenous catalyst is located on a cell. In some embodiments, the endogenous catalyst is located on a blood cell. In some embodiments, the endogenous catalyst is located in a cell. In some embodiments, the endogenous catalyst is secreted by a blood cell. In some embodiments, the endogenous catalyst is located on a cell. In some embodiments, the endogenous catalyst is secreted by a blood cell.

In some embodiments, the monomer is a catechol-based monomer. In some embodiments, the monomer comprises a 1,2-dihydroxybenzene moiety. In some embodiments, the monomer comprises an optionally substituted 1,2-dihydroxybenzene moiety. In some embodiments, the monomer comprises 1,2-dihydroxyphenyl. In some embodiments, the monomer comprises an optionally substituted 1,2-dihydroxyphenyl.

In some embodiments, the monomer is a 2-(3,4-dihydroxyphenyl)ethylamine-based monomer. In certain embodiments, the monomer comprises a 3,4-dihydroxyphenethylamine moiety. In certain embodiments, the monomer comprises an optionally substituted 3,4-dihydroxyphenethylamine moiety. In certain embodiments, the monomer comprises 2-(3,4-dihydroxyphenyl)ethylamine. In certain embodiments, the monomer comprises an optionally substituted 2-(3,4-dihydroxyphenyl)ethylamine moiety.

In some embodiments, the monomer is selected from the group consisting of dopamine, levodopa, norepinephrine, methyldopa, levodopa methyl ester, levodopa ethyl ester, derivatives thereof, and combinations thereof. In certain embodiments, the monomer is selected from the group consisting of dopamine, levodopa, norepinephrine, methyldopa, levodopa methyl ester, and levodopa ethyl ester.

In certain embodiments, the monomer is a derivative of dopamine. In some embodiments, the monomer comprises dopamine, levodopa, norepinephrine, methyldopa, levodopa methyl ester, or levodopa ethyl ester.

In some embodiments, the derivative of the monomer comprises a macromolecule. In some embodiments, the monomer comprises dopamine, levodopa, norepinephrine, methyldopa, levodopa methyl ester, or levodopa ethyl ester and at least one macromolecule. In certain embodiments the macromolecule is alginate, hyaluronic acid, polyacrylic acid, polyethylene glycol, chondroitin sulfate, chitosan, or a combination thereof. In some embodiments the macromolecule is alginate, hyaluronic acid, polyacrylic acid, polyethylene glycol, chondroitin sulfate, or chitosan. In certain embodiments the monomer comprises (A) dopamine, levodopa, norepinephrine, methyldopa, levodopa methyl ester, or levodopa ethyl ester moieties bound to (B) alginate, hyaluronic acid, polyacrylic acid, polyethylene glycol, chondroitin sulfate, or chitosan. In certain embodiments the monomer comprises (A) more than one of dopamine, levodopa, norepinephrine, methyldopa, levodopa methyl ester, and levodopa ethyl ester moieties bound to (B) alginate, hyaluronic acid, polyacrylic acid, polyethylene glycol, chondroitin sulfate, or chitosan. In certain embodiments the monomer comprises (A) more than one of dopamine, levodopa, norepinephrine, methyldopa, levodopa methyl ester, and levodopa ethyl ester moieties bound to (B) more than one of alginate, hyaluronic acid, polyacrylic acid, polyethylene glycol, chondroitin sulfate, and chitosan.

In certain embodiments, the monomer is selected from the structures listed in Table 1 or 2.

TABLE 1
Monomers

| Monomer | Structure |
|---|---|
| M1 | 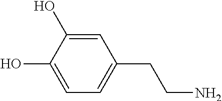 [SMILES: NCCc1ccc(O)c(O)c1] |
| M2 | 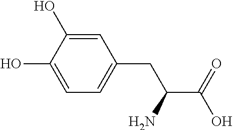 [SMILES: N[C@@H](Cc1ccc(O)c(O)c1)C(=O)O] |
| M3 | 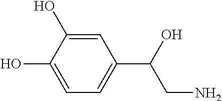 [SMILES: NCC(O)c1ccc(O)c(O)c1] |
| M4 | 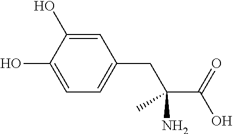 |
| M5 | 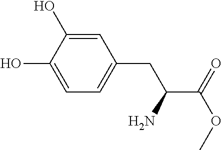 |
| M6 | 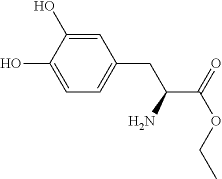 [SMILES: CCOC(=O)[C@@H](N)Cc1ccc(O)c(O)c1] |

TABLE 2
Macromolecules

| Macromolecule | Components | Structure |
|---|---|---|
| C1 | M1 and alginate | 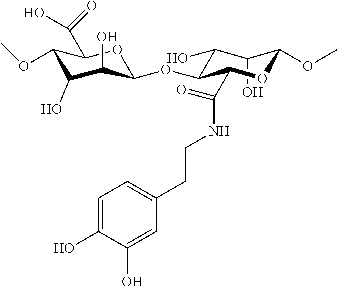 |
| C2 | M2 and alginate | 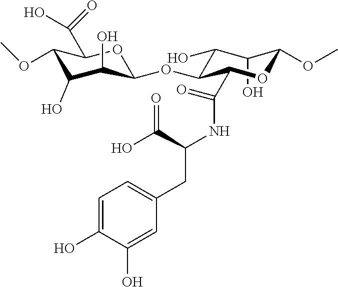 |
| C3 | M3 and alginate | 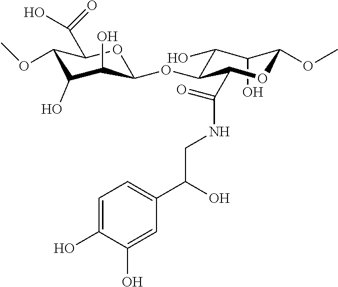 |
| C4 | M4 and alginate | 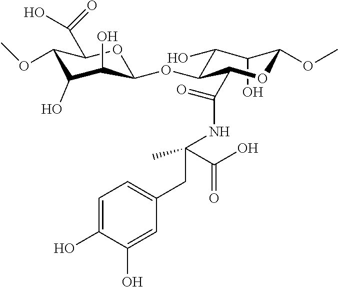 |
| C5 | M5 and alginate | 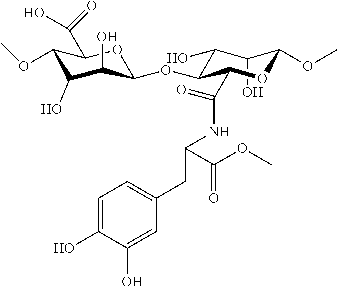 |
| C6 | M6 and alginate | 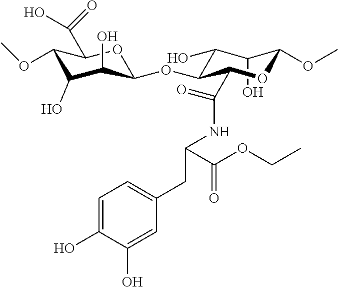 |
| C7 | M1 and hyaluronic acid | 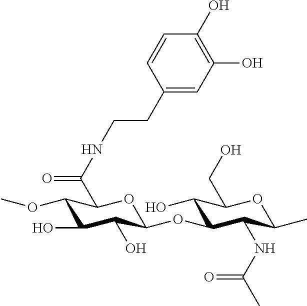 |
| C8 | M2 and hyaluronic acid | 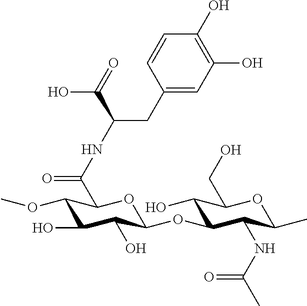 |
| C9 | M3 and hyaluronic acid | 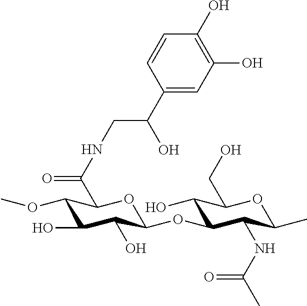 |
| C10 | M4 and hyaluronic acid | 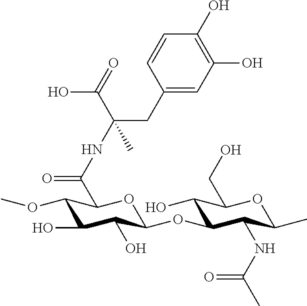 |
| C11 | M5 and hyaluronic acid | 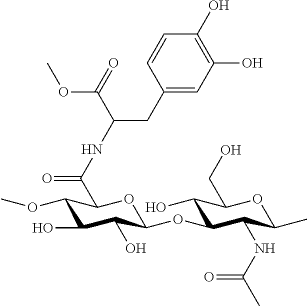 |
| C12 | M6 and hyaluronic acid | 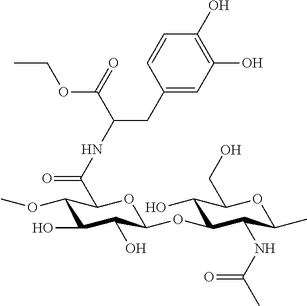 |
| C13 | M1 and polyacrylic acid | 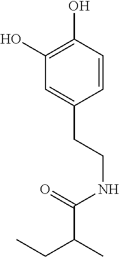 [SMILES: CCC(C)C(=O)NCCc1ccc(O)c(O)c1] |
| C14 | M2 and polyacrylic acid | 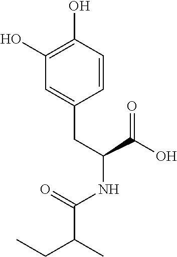 [SMILES: CCC(C)C(=O)N[C@@H](Cc1ccc(O)c(O)c1)C(=O)O] |
| C15 | M3 and polyacrylic acid | 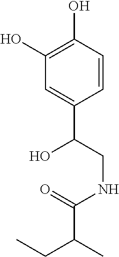 [SMILES: CCC(C)C(=O)NCC(O)c1ccc(O)c(O)c1] |
| C16 | M4 and polyacrylic acid | 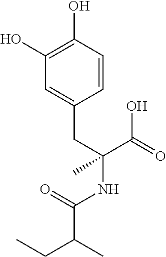 [SMILES: CCC(C)C(=O)N[C@@](C)(Cc1ccc(O)c(O)c1)C(=O)O] |
| C17 | M5 and polyacrylic acid | 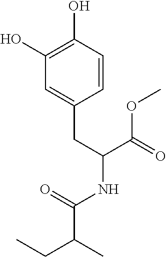 [SMILES: CCC(C)C(=O)NC(Cc1ccc(O)c(O)c1)C(=O)OC] |
| C18 | M6 and polyacrylic acid | 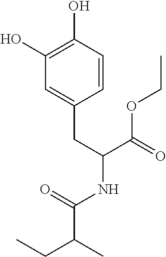 [SMILES: CCOC(=O)C(Cc1ccc(O)c(O)c1)NC(=O)C(C)CC] |
| C19 | M1 and polyethylene with tripentaerythritol core | 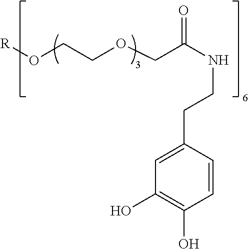 |
| | | R = tripentaerythritol core structure |
| C20 | M1 and chondroitin sulfate | 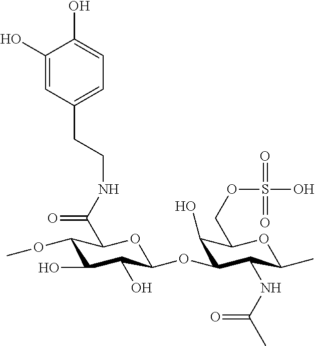 |
| C21 | M1 and chitosan | 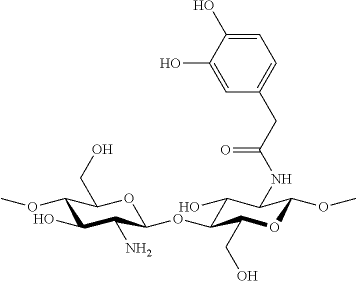 |

In some embodiments, the monomer consists of a single type of monomer. In certain embodiments, the monomer comprises a combination of monomers. In certain embodiments, the monomer comprises, a combination of monomers listed in Tables 1 and 2. In some embodiments, the combination of monomers consists of two different monomers. In some embodiments, the combination of monomers consists of three different monomers. In some embodiments, the combination of monomers consists of four different monomers.

In some embodiments, the composition further comprises an agent selected from active pharmaceutical agents, cosmetic agents, nutraceutical agents, imaging agents, diagnostic agents, radioprotective agents, nutraceutical agents, enzymes, nutrient blockers, aptamers, antibodies, neutralizing agents, and combinations thereof. active pharmaceutical agent In some embodiments, the composition further comprises an enzyme. In some embodiments, the composition further comprises a nutrient blocker. In some embodiments, the composition further comprises a radioprotective agent. In some embodiments, the composition further comprises an active pharmaceutical agent. In some embodiments, the composition further comprises a diagnostic agent. In some embodiments, the composition further comprises a combination of two or more of enzymes, nutrient blockers, radioprotective agents, active pharmaceutical agents, and diagnostic agents.

In certain embodiments, the composition further comprises a buffer.

In some embodiments, the composition further comprises an exogenous catalyst.

In some embodiments, at least one of the monomer and the oxygen source is stable in the stomach of the subject. In some embodiments, at least one of the monomer and the oxygen source is stable in the stomach for at least 30 minutes of the subject. In some embodiments, at least one of the monomer and the oxygen source is stable in the stomach for at least 60 minutes of the subject.

In some embodiments, the composition is stable in the stomach of the subject. In some embodiments, the composition is stable in the stomach for at least 30 minutes of the subject. In some embodiments, the composition is stable in the stomach for at least 60 minutes of the subject.

In some embodiments, at least one of the monomer and the oxygen source is stable in that it does not decompose in the stomach of the subject. In some embodiments, at least 95% of at least one of the monomer and the oxygen source remains after the composition passes out of the stomach of the subject. In some embodiments, at least 90% of at least one of the monomer and the oxygen source remains after the composition passes out of the stomach of the subject. In some embodiments, at least 80% of at least one of the monomer and the oxygen source remains after the composition passes out of the stomach of the subject.

In some embodiments, the composition comprises about 0.001 to about 1000 mg/mL monomer. In some embodiments, the composition comprises about 0.01 to about 100 mg/mL of monomer. In some embodiments, the composition comprises about 0.01 to about 50 mg/mL of monomer. In some embodiments, the composition comprises about 1 to about 20 mg/mL of monomer. In some embodiments, the composition comprises 10 mg/mL of monomer. In some embodiments, the composition comprises 20 mg/mL of monomer.

In some embodiments, the composition comprises about 0.01 to about 100 mM of the oxygen source. In some embodiments, the composition comprises about 0.1 to about 50 mM of the oxygen source. In some embodiments, the composition comprises about 1 to about 30 mM of the oxygen source. In some embodiments, the composition comprises about 20 mM of the oxygen source.

In some embodiments, the composition comprises a concentration of oxygen source compatible with ingestion by the subject.

In some embodiments, the composition has a pH of about 7 to about 10. In some embodiments, the composition has a pH of about 7 to about 9. In some embodiments, the composition has a pH of about 8.5. In some embodiments, the composition has a pH of about 7.4.

In certain embodiments, the composition is administered directly to the target site of polymerization. In some embodiments, the composition is directly applied to the site of polymerization. In certain embodiments, the composition is applied to eye wherein polymerization occurs. In some embodiments, the composition is administered indirectly to the target site of polymerization. In certain embodiments, the composition is administered orally and the site of polymerization is within the GI tract. In some embodiments, the composition is administered by intra-articular injection and the site of polymerization is within a joint.

In some embodiments, the composition is administered by a route selected from oral, rectal, injection, sublingual, buccal, vaginal, ocular, otic, inhalation, or cutaneous. In some embodiments, the composition is administered orally. In certain embodiments, the composition is administered by intra-articular injection. In certain embodiments, the composition is administered topically. In certain embodiments, the composition is administered dermally. In certain embodiments, the composition is administered ophthalmically.

In some embodiments, the composition is a liquid or a solid dosage form.

In some embodiments, the composition is in the form of a solution, a gel, a tablet, a powder, a capsule, eye drops, foam, a transdermal patch, or combinations thereof. In some embodiments, the composition is in the form of a solution, a gel, a tablet, or a capsule. In some embodiments, the composition is in the form of a solution. In some embodiments, the composition is in the form of a solution. In some embodiments, the composition is in the form of eye drops. In some embodiments, the composition is in the form of a powder. In some embodiments, the composition is in the form of a transdermal patch.

In some embodiments, the polymer adheres to a tissue of the subject. In certain embodiments, the polymer forms in contact with and adheres to a tissue in the subject. In some embodiments, the polymer adheres to a tissue of the subject

In some embodiments, the location of polymer formation is based on expression levels of the catalyst. In certain embodiments, the polymer forms substantially on a particular tissue based on high expression levels of catalyst. In some embodiments, the polymer does not substantially form on a particular tissue due to low expression levels of catalyst. In some embodiments, the location of polymer formation is based on expression levels of catalase. In certain embodiments, the polymer forms substantially on a particular tissue based on expression levels of catalase. In some embodiments, the polymer does not substantially form on a particular tissue due to low expression levels of catalase.

In certain embodiments, the polymer forms on and adheres to tissue in one or more of the respiratory, urinary, lymphatic, circulatory, integumentary system, skeletal, muscular, nervous, digestive, or endocrine systems. In certain embodiments, the polymer forms in contact with and adheres to one or more of the dorsal portion of the tongue, buccal mucosa, labial mucosa, palate, nasal mucosa, conjunctiva, hypodermis, muscle, trachea mucosa, lung surface, lung tissue, gallbladder mucosa, gallbladder serosa, tunica intima, cartilage, lymph node, bladder mucosa, bladder serosa, kidney surface, kidney tissue, endocardium, myocardium, pancreas tissue, nerve, liver surface, liver tissue, spleen tissue, small intestine, colon, ventral tongue, sclera, epidermis, bladder serosa, epicardium, liver surface, esophagus, peritoneum, or stomach. In some embodiments, the polymer forms in or on the dorsal portion of the tongue, buccal mucosa, labial mucosa, palate, nasal mucosa, conjunctiva, hypodermis, muscle, trachea mucosa, lung surface, lung tissue, gallbladder mucosa, gallbladder serosa, tunica intima, cartilage, lymph node, bladder mucosa, bladder serosa, kidney surface, kidney tissue, endocardium, myocardium, pancreas tissue, nerve, liver surface, liver tissue, spleen tissue, small intestine, or colon. In some embodiments, the polymer forms in or on the trachea, lung, kidney, bladder, lymph node, heart, blood vessel, skin, cartilage, muscle, nerve, mouth, liver, spleen, gallbladder, pancreas, or small bowel. In certain embodiments, the polymer forms on one or more of the tongue, the buccal mucosa, and the labial mucosa. In some embodiments, the polymer forms on the conjunctiva of the eye. In some embodiments, the polymer forms on cartilage. In some embodiments, polymer forms on cartilage in a joint.

In some embodiments, the polymer does not substantially form on the ventral site of the tongue. In some embodiments, the polymer does not substantially form on the palate. In some embodiments, the polymer does not substantially form on the sclera. In some embodiments, the polymer does not substantially form on the ventral portion of the tongue, sclera, epidermis, bladder serosa, epicardium, liver surface, esophagus, or stomach. In some embodiments, the polymer does not substantially form on the ventral portion of the tongue, sclera, epidermis, epicardium, esophagus, or stomach.

In certain embodiments, the tissue is epithelium. In some embodiments, the epithelium is intestinal epithelium. In some embodiments, the polymer forms in contact with the epithelium of the subject. In some embodiments, the epithelium is intestinal epithelium. In some embodiments, the polymer forms on the small intestine. In certain embodiments, the polymer forms in the lumen of the small intestine. In certain embodiments, the polymer forms on the epithelium of the duodenum of the subject.

In some embodiments, the polymer forms on the epithelium of the gastrointestinal tract of the subject. In some embodiments, the polymer forms on the epithelium of the small intestine of the subject.

In some embodiments, the polymer does not form on the epithelium of the gastrointestinal tract outside the small intestine of the subject.

In some embodiments, the polymer forms on the epithelium of one or more of the duodenum, the jejunum, the ileum, the colon, the esophagus, or the stomach of the subject. In some embodiments, the polymer forms on the epithelium of the duodenum of the subject. In some embodiments, the polymer forms on the epithelium of the jejunum of the subject. In some embodiments, the polymer forms on the epithelium of the ileum of the subject. In some embodiments, the polymer forms on the epithelium of the colon of the subject.

In some embodiments, the polymer does not substantially form on the epithelium of one or more of the esophagus or stomach of the subject. In some embodiments, substantially no polymer forms on the stomach and the esophagus of the subject. In some embodiments, polymer does not form on the stomach and the esophagus of the subject.

In some embodiments, less is polymer formed on the ileum and the colon of the subject as compared to the duodenum and the jejunum of the subject.

In some embodiments, the polymer forms on the villi of the epithelium of the subject.

In some embodiments, the polymer binds with chemical moieties exposed on the surface of tissue in the respiratory, urinary, lymphatic, circulatory, integumentary system, skeletal, muscular, nervous, digestive, or endocrine system. In some embodiments, the polymer binds with amine moieties exposed on the surface of tissue in the respiratory, urinary, lymphatic, circulatory, integumentary system, skeletal, muscular, nervous, digestive, or endocrine system.

In some embodiments, the polymer binds with amine moieties exposed on the luminal surface of the epithelium of the subject. In some embodiments, the polymer crosslinks with amine moieties exposed on the luminal surface of the epithelium of the subject.

In some embodiments, the polymer is rapidly formed. In some embodiments, the polymer is formed in less than about 20 minutes. In some embodiments, the polymer is formed in less than about 15 minutes. In some embodiments, the polymer is formed in less than about 12 minutes. In some embodiments, the polymer is formed in less than about 10 minutes. In some embodiments, the polymer is formed in less than about 5 minutes. In some embodiments, the polymer is formed in less than about 3 minutes. In some embodiments, the polymer is formed in less than about 2 minutes. In some embodiments, the polymer is formed in less than about 1 minute. In some embodiments, the polymer forms almost instantly.

In some embodiments, the rate of polymerization increases by at least 10 times as compared to polymer formation without endogenous catalase. In some embodiments, the rate of polymerization increases by at least 50 times as compared to polymer formation without endogenous catalase. In some embodiments, the rate of polymerization increases by at least 100 times as compared to polymer formation without endogenous catalase. In some embodiments, the rate of polymerization increases by at least 150 times as compared to polymer formation without endogenous catalase. In some embodiments, the rate of polymerization increases by at least 200 times as compared to polymer formation without endogenous catalase.

In some embodiments, the polymer forms a temporary barrier in vivo. In some embodiments, the polymer forms a transient barrier in vivo.

In some embodiments, the polymer lasts for about 30 minutes. In some embodiments, the polymer lasts for about 1 hour. In some embodiments, the polymer lasts for about 6 hours. In some embodiments, the polymer lasts for about 12 hours. In some embodiments, the polymer lasts for about 24 hours.

In some embodiments, about 20 to about 70% of the transient barrier remains after 12 hours. In some embodiments, about 30 to about 50% of the transient barrier remains after 12 hours. In some embodiments, about 20% of the transient barrier remains after 12 hours. In some embodiments, about 20 to about 70% of the transient barrier remains after 6 hours. In some embodiments, about 30 to about 50% of the transient barrier remains after 6 hours. In some embodiments, about 20% of the transient barrier remains after 6 hours. In some embodiments, about 20 to about 70% of the transient barrier remains after 3 hours. In some embodiments, about 30 to about 50% of the transient barrier remains after 3 hours. In some embodiments, about 20% of the transient barrier remains after 3 hours.

In some embodiments, the polymer is cleared from the subject after about 3 hours. In some embodiments, the polymer is cleared from the subject after about 6 hours. In some embodiments, the polymer is cleared from the subject after about 12 hours. In some embodiments, the polymer is cleared from the subject after about 24 hours. In some embodiments, the polymer is cleared from the subject after about 48 hours.

In some embodiments, the polymer barrier provides for selective molecular transport in the patient.

In some embodiments, the method is a method of modulating diffusion in the subject at the site of polymerization. In some embodiments, the method modulates diffusion of one or more of a salt, an ion, water, oxygen, carbon dioxide, carbonate anion, an acid, a base, a carbohydrate, a lipid, a protein, a nucleic acid, a nutrient, or an active pharmaceutical agent in the subject.

In some embodiments, polymer modulates absorption of one or more nutrients or active pharmaceutical agents within the small intestine.

In some embodiments, polymer substantially impedes absorption of one or more nutrients to the epithelium on which the polymer is formed, to the intestinal wall of the subject, or to the blood stream of the subject.

In some embodiments, the method is a method of delivering an agent to the subject. In some embodiments, the agent is an active pharmaceutical agent. In some embodiments, the agent is an enzyme. In some embodiments, the agent is a radioprotective agent.

In some embodiments, the method enables sustained release of the agent in the subject. In some embodiments, the agent is an active pharmaceutical agent. In some embodiments, the agent is an enzyme. In some embodiments, the agent is a radioprotective agent. In certain embodiments, the method enables sustained release of an active pharmaceutical agent in the eye conjunctiva. In certain embodiments, n the method enables sustained release of an active pharmaceutical agent in cartilage. In certain embodiments, the method enables sustained release of an active pharmaceutical agent in the oral cavity.

In some embodiments, the method is a method of immobilizing an agent in a subject. In some embodiments, the agent is an active pharmaceutical agent. In some embodiments, the agent is an enzyme. In some embodiments, the agent is a radioprotective agent.

In some embodiments, the method is a method of localized delivery of an agent in a subject. In some embodiments, the agent is an active pharmaceutical agent. In some embodiments, the agent is an enzyme. In some embodiments, the agent is a radioprotective agent.

In some embodiments, the method is a method of reducing the dosing frequency of the agent. In some embodiments, the agent is an active pharmaceutical agent. In some embodiments, the agent is an enzyme. In some embodiments, the agent is a radioprotective agent.

In some embodiments, the method is a method of increasing the half-life of the agent in the subject. In some embodiments, the agent is an active pharmaceutical agent. In some embodiments, the agent is an enzyme. In some embodiments, the agent is a radioprotective agent.

In some embodiments, the method is a method of increasing residence time of the agent in the subject. In some embodiments, the agent is an active pharmaceutical agent. In some embodiments, the agent is an enzyme. In some embodiments, the agent is a radioprotective agent.

In some embodiments, the method is a method of treating or preventing a disease in a subject. In some embodiments, the method is a method of treating a disease in a subject. In some embodiments, the method is a method of preventing a disease in a subject.

In some embodiments, the method is a method of aiding in tissue recovery and regeneration in the subject at the site of polymerization. In some embodiments, the method is a method of aiding in tissue recovery in the subject at the site of polymerization. In some embodiments, the method is a method of aiding in tissue regeneration in the subject at the site of polymerization.

In some embodiments, the method causes the intestinal lumen of the subject to remain expanded.

In some embodiments, the method is a method of preventing a bowel adhesion in the subject.

In some embodiments, the method is a method of preventing a bowel obstruction in the subject.

In some embodiments, the method is a method of treating bleeding in the subject. In some embodiments, bleeding is in the upper GI tract.

In some embodiments, the polymer and composition further comprises an enzyme. In some embodiments, the enzyme is a digestive enzyme. In some embodiments, the digestive enzyme is lactase, peptidase, sucrase, maltase, amylase, a lipase, or a protease. In some embodiments, the digestive enzyme is β-galactosidase.

In some embodiments, the method is a method of improving digestion efficiency by the subject.

In some embodiments, the method is a method of augmenting digestion of a sugar by the subject. In some embodiments, the method is a method of augmenting digestion of lactose by the subject.

In some embodiments, the method is a method of treating lactose intolerance in the subject.

In some embodiments, the enzyme improves digestion efficiency of a sugar of the subject by about 5 times. In some embodiments, the enzyme improves digestion efficiency of a sugar of the subject by about 10 times. In some embodiments, the enzyme improves digestion efficiency of a sugar of the subject by about 20 times. In some embodiments, the enzyme improves digestion efficiency of lactose of the subject by about 40 times. In some embodiments, the enzyme improves digestion efficiency of a sugar of the subject by about 50 times.

In some embodiments. β-galactosidase improves digestion efficiency of lactose of the subject by about 5 times. In some embodiments, (i-galactosidase improves digestion efficiency of lactose of the subject by about 10 times. In some embodiments, β-galactosidase improves digestion efficiency of lactose of the subject by about 20 times. In some embodiments, β-galactosidase improves digestion efficiency of lactose of the subject by about 40 times. In some embodiments, β-galactosidase improves digestion efficiency of lactose of the subject by about 50 times.

In some embodiments, the polymer barrier does not inhibit the intrinsic digestive enzyme activity of the epithelium of the subject.

In some embodiments, the polymer and composition further comprises a nutrient blocker.

In some embodiments, the method is a method of preventing nutrient absorption in the subject.

In some embodiments, the method is a method of modulating or regulating sugar absorption by the subject. In some embodiments, the sugar is selected from glucose, lactose, fructose, maltose, dextrose, galactose, sucrose, and isomaltose. In some embodiments, the method is a method of modulating or regulating glucose absorption by the subject.

In some embodiments, the method prevents absorption for less than about 48 hours. In some embodiments, the method prevents absorption for less than about 24 hours. In some embodiments, the method prevents absorption for less than about 12 hours. In some embodiments, the method prevents absorption for less than about 6 hours. In some embodiments, the method prevents absorption for less than about 3 hours.

In some embodiments, the method is a method of treating obesity in the subject.

In some embodiments, the method is a method of treating hyperinsulinemia in the subject.

In some embodiments, the method is a method of treating diabetes mellitus in the subject. In some embodiments, the diabetes mellitus is type 2 diabetes mellitus.

In some embodiments, the method is a method of treating non-alcoholic fatty liver disease. In some embodiments, the method is a method of treating nonalcoholic steatohepatitis.

In some embodiments, the composition further comprises a crosslinking agent.

In some embodiments, the crosslinking agent comprises a nanoparticle. In some embodiments, the crosslinking agent comprises polydopamine. In some embodiments, the crosslinking agent is a nutrient blocker. In some embodiments, the crosslinking agent improves the nutrient blocking ability of the polymer.

In some embodiments, glucose absorption is modulated by tuning the crosslinking density of polymer.

In some embodiments, the method reduces glucose absorption by the subject by at least about 50%, at least about 60%, or at least about 70% for a period of 3 hours following administration of the composition. In some embodiments, the method reduces glucose absorption by the subject by at least about 70% for a period of 3 hours following administration of the composition. In some embodiments, the method reduces glucose absorption by the subject by at least about 50%, at least about 60%, or at least about 70% for a period of 2 hours following administration of the composition. In some embodiments, the method reduces glucose absorption by the subject by at least about 50%, at least about 60%, or at least about 70% for a period of 1 hours following administration of the composition.

In some embodiments, the method is a method of regulating or modulating nutrient uptake by the subject.

In some embodiments, the composition further comprises an active pharmaceutical agent. In some embodiments, the active pharmaceutical agent treats an infectious disease. In some embodiments, the active pharmaceutical agent is an antiparasitic drug. In some embodiments, the active pharmaceutical agent is an anthelmintic drug. In some embodiments, the active pharmaceutical agent is praziquantel. In some embodiments, the active pharmaceutical agent is an antiviral drug. In some embodiments, the active pharmaceutical agent treats influenza. In some embodiments, the active pharmaceutical agent treats SARS-CoV-2.

In certain embodiments, the active pharmaceutical ingredient is a contraceptive, a statin, an anti-hypertensive, or an antibiotic.

In some embodiments, the active pharmaceutical agent treats psychiatric disorders, Alzheimer's disease, infection diseases, or transplant rejection.

In some embodiments, the active pharmaceutical agent treats type 2 diabetes.

In some embodiments, the active pharmaceutical agent treats non-alcoholic fatty liver disease. In some embodiments, the active pharmaceutical agent treats nonalcoholic steatohepatitis.

In some embodiments, the active pharmaceutical agent treats ocular diseases.

In some embodiments, the active pharmaceutical agent treats Crohn's disease.

In some embodiments, the active pharmaceutical agent treats osteoarthritis.

In some embodiments, the active pharmaceutical agent treats Alzheimer's disease.

In some embodiments, the active pharmaceutical agent is an anti-cancer agent. Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon α), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1. AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121. XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the active pharmaceutical agent is selected from the group including, but not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the active pharmaceutical agent is an anti-proliferative agent. In certain embodiments, the active pharmaceutical agent is an anti-cancer agent. In certain embodiments, the active pharmaceutical agent is an anti-viral agent.

Exemplary active pharmaceutical agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins, etc. Active pharmaceutical agent include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, the active pharmaceutical agent is an antibiotic. Exemplary antibiotics include, but are not limited to, penicillins (e.g., penicillin, amoxicillin), cephalosporins (e.g., cephalexin), macrolides (e.g., erythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aureomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, quinupristin/dalfoprisin (Syndercid™). In certain embodiments, the antibiotic is a ribosome-targeting antibiotic.

In some embodiments, the active pharmaceutical agent is retained or encapsulated in the polymer. In some embodiments, the active pharmaceutical agent is retained on the polymer.

In some embodiments, the method is a method of prolonging the residence time of an active pharmaceutical agent in the subject as compared to the administration of the active pharmaceutical agent in the absence of the polymer. In some embodiments, the method is a method of prolonging the residence time of an active pharmaceutical agent at the site of polymerization in the subject as compared to the administration of the active pharmaceutical agent in the absence of the polymer.

In some embodiments, the method is a method of providing for sustained release of an active pharmaceutical agent as compared to the administration of the active pharmaceutical agent in the absence of the polymer.

In some embodiments, the method is a method of reducing the dosing frequency of an active pharmaceutical agent as compared to the administration of the active pharmaceutical agent in the absence of the polymer. In some embodiments, the dosing frequency is once per day. In some embodiments, the dosing frequency is twice per day.

In some embodiments, the method is a method of increasing the half-life of the active pharmaceutical agent as compared to the administration of the active pharmaceutical agent in the absence of the polymer. In some embodiments, the method increases the half-life of the active pharmaceutical agent by at least about 2-fold as compared to the administration of the active pharmaceutical agent in the absence of the polymer. In some embodiments, the method increases the half-life of the active pharmaceutical agent by at least about 4-fold as compared to the administration of the active pharmaceutical agent in the absence of the polymer. In some embodiments, the method increases the half-life of the active pharmaceutical agent by at least about 6-fold as compared to the administration of the active pharmaceutical agent in the absence of the polymer. In some embodiments, the method increases the half-life of the active pharmaceutical agent by at least about 10-fold as compared to the administration of the active pharmaceutical agent in the absence of the polymer.

In some embodiments, the method is a method of increasing the AUC of the active pharmaceutical agent as compared to the administration of the active pharmaceutical agent in the absence of the polymer. In some embodiments, the method increases the AUC by at least about 2-fold as compared to the administration of the active pharmaceutical agent in the absence of the polymer. In some embodiments, the method increases the AUC by at least about 3-fold as compared to the administration of the active pharmaceutical agent in the absence of the polymer. In some embodiments, the method increases the AUC by at least about 4-fold as compared to the administration of the active pharmaceutical agent in the absence of the polymer.

In some embodiments, the method is a method of modulating the Cₘₐₓ of the active pharmaceutical agent as compared to the administration of the active pharmaceutical agent in the absence of the polymer. In certain embodiments, the method of modulating is increasing. In certain embodiments, the method of modulating is decreasing.

In some embodiments, the method is a method of modulating the Tₘₐₓ of the active pharmaceutical agent as compared to the administration of the active pharmaceutical agent in the absence of the polymer. In certain embodiments, the method of modulating is increasing. In certain embodiments, the method of modulating is decreasing.

In some embodiments, the method does not affect drug metabolism once the active pharmaceutical agent is absorbed by the small intestine.

In some embodiments, the method is a method of treating schistosomiasis in the subject.

In some embodiments, the composition further comprises a radioprotective agent. In certain embodiments, the radioprotective agent is an antioxidant, a thiol-containing compound, or a nitroxide. In certain embodiments, the radioprotective agent is thalidomide, cysteine, amifostine, palifermin, or 1-carnitine. In certain embodiments, the radioprotective agent is thalidomide.

In some embodiments, the nutraceutical agent is vitamin D or iron.

In some embodiments, the method provides for targeting the small intestine.

In some embodiments, the method is a method of decreasing uptake by the small intestine. In some embodiments, the method is a method of decreasing uptake of one or more nutrients and active pharmaceutical agents by the small intestine. In some embodiments, the method is a method of decreasing uptake of one or more nutrients by the small intestine. In some embodiments, the method is a method of decreasing uptake of one or more active pharmaceutical agents by the small intestine.

In some embodiments, the method is a method of increasing residence time in the small intestine. In some embodiments, the method is a method of increasing residence time of one or more of nutrients and active pharmaceutical agents in the small intestine. In some embodiments, the method is a method of increasing residence time of one or more of nutrients in the small intestine. In some embodiments, the method is a method of increasing residence time of one or more active pharmaceutical agents in the small intestine.

In some embodiments, the method causes the intestinal lumen to remain expanded.

In some embodiments, the method is a method of treating or preventing a bowel adhesion in the subject. In some embodiments, the method is a method of preventing a bowel adhesion in the subject.

In some embodiments, the method is a method of treating or preventing bowel obstruction in the subject. In some embodiments, the method is a method of preventing bowel obstruction in the subject.

In some embodiments, the method is a method of treating bleeding in the subject. In some embodiments, the method is a method of treating bleeding in the small intestine of the subject. In some embodiments the bleeding is in the upper GI tract. In some embodiments, the bleeding is in the stomach. In some embodiments, the method of treating bleeding is a method of treating hemostasis.

In some embodiments, the polymer modulates absorption within the small intestine. In some embodiments, the polymer modulates absorption of one or more nutrients or active pharmaceutical agents, or combinations thereof, within the small intestine. In some embodiments, the polymer modulates absorption of one or more nutrients and active pharmaceutical agents within the small intestine. In some embodiments, the polymer modulates absorption of one or more nutrients within the small intestine. In some embodiments, the polymer modulates absorption of one or more active pharmaceutical agents within the small intestine.

In some embodiments, the polymer modulates digestion within the small intestine. In some embodiments, the polymer modulates digestion of one or more nutrients within the small intestine.

In certain embodiments, the polymer substantially impedes absorption by the small intestine. In some embodiments, the polymer substantially impedes absorption of one or more nutrients or active pharmaceutical agents, or combinations thereof, to the epithelium on which the polymer is formed. In some embodiments, the polymer substantially impedes absorption of one or more nutrients to the epithelium on which the polymer is formed. In some embodiments, the polymer substantially impedes absorption of one or more active pharmaceutical agents to the epithelium on which the polymer is formed.

In some embodiments, the polymer substantially impedes absorption of one or more nutrients or active pharmaceutical agents, or combinations thereof, to the intestinal wall of the subject. In some embodiments, the polymer substantially impedes absorption of one or more nutrients to the intestinal wall of the subject. In some embodiments, the polymer substantially impedes absorption of one or more active pharmaceutical agents to the intestinal wall of the subject.

In some embodiments, the polymer substantially impedes absorption of one or more nutrients or active pharmaceutical agents, or combinations thereof, to the blood stream of the subject. In some embodiments, the polymer substantially impedes absorption of one or more nutrients to the blood stream of the subject. In some embodiments, the polymer substantially impedes absorption of one or more active pharmaceutical agents to the blood stream of the subject.

In some embodiments, the method is a method of immobilizing an enzyme in a subject.

In some embodiments, the method is a method of delivering an active pharmaceutical agent to the subject.

In some embodiments, the method is a method of supplementing the digestion in a subject.

In some embodiments, the polymer induces blood gelation. In certain embodiments, the polymer induces coagulation. In some embodiments, the composition is in the form of a powder. In certain embodiments, the method comprises spraying the powder onto an affected area on or within the subject.

In some embodiments, the composition is administered via a scope. In certain embodiments, the composition is administered via an endoscope, arthroscope, cystoscope, colposcope, colonoscope, bronchoscope, ureteroscope, anoscope, esophagoscope, gastroscope, laparoscope, laryngoscope, neuroendoscope, proctoscope, sigmoidoscope, or thoracoscope.

In some embodiments, the polymer is nontoxic. In some embodiments, the composition is nontoxic. In some embodiments, the composition and its components are nontoxic.

In some embodiments, the polymer is stable to physical and chemical forces. In some embodiments, the polymer is stable to one or more of intestinal fluid, intestinal acid, gastric acid, chyme, ethanol, and saline. In some embodiments, the polymer decomposes by less than about 25% upon exposure to one or more of intestinal fluid, intestinal acid, gastric acid, chyme, ethanol, or saline. In some embodiments, the polymer decomposes by less than about 20% upon exposure to one or more of intestinal fluid, intestinal acid, gastric acid, chyme, ethanol, or saline. In some embodiments, the polymer decomposes by less than about 10% upon exposure to one or more of intestinal fluid, intestinal acid, gastric acid, chyme, ethanol, or saline. In some embodiments, the polymer decomposes by less than about 5% upon exposure to one or more of intestinal fluid, intestinal acid, gastric acid, chyme, ethanol, or saline. In some embodiments, the physical forces are selected from one or more of peristalsis and segmentation.

In another aspect, the disclosure provides a method of treating a disease or disorder comprising administering an effective amount of a composition as described herein to a subject in need thereof.

Further provided by the disclosure is a method of preventing a disease or disorder comprising administering an effective amount of a composition as described herein to a subject in need thereof.

In some embodiments, the disease or disorder is an eye disease, a joint disease, a metabolic disorder, a systemic disease, a digestive disorder, cancer, bleeding, an ulcer, a bowel obstruction, an infectious disease, mesenteric ischemia, obesity, a psychiatric disorder, Alzheimer's disease, or transplant rejection. In some embodiments, the disease or disorder is an eye disease, a joint disease, a metabolic disorder, a systemic disease, a digestive disease, cancer, bleeding, an ulcer, a bowel obstruction, an infectious disease, mesenteric ischemia, or obesity. In some embodiments, the disease or disorder is a psychiatric disorder, Alzheimer's disease, or transplant rejection.

In some embodiments, the disease is cancer. Exemplary cancers include, but are not limited to, acoustic neuroma; adenocarcinoma; adrenal gland cancer, anal cancer; angiosarcoma (e.g., lymphangiosarcoma, lymphangioendotheliosarcoma, hemangiosarcoma); appendix cancer, benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor, cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer, epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer, inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer, rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer, and vulvar cancer (e.g., Paget's disease of the vulva).

In some embodiments, the disease is glaucoma.

In some embodiments, the metabolic disorder is hyperinsulinemia.

In some embodiments, the digestive disease is Crohn's disease, ulcerative colitis, malabsorption, inflammatory bowel disease, irritable bowel syndrome, lactose intolerance, or Celiac disease. In some embodiments, the disease is Crohn's disease.

In some embodiments, the disease is a psychiatric disorder.

In some embodiments, the disease is Alzheimer's disease.

In some embodiments, the disorder is transplant rejection.

In some embodiments, the systemic disease is an autoimmune disease. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In some embodiments, the systemic disease is mastocytosis, chronic fatigue syndrome, systemic vasculitis, sarcoidosis, hypothyroidism, diabetes, fibromyalgia, adrenal insufficiency, celiac disease, ulcerative colitis, Crohn's disease, hypertension, metabolic syndrome, AIDS, Graves' disease, systemic lupus erythematosus, arthritis, atherosclerosis, sickle cell disease, myasthenia gravis, systemic sclerosis, inflammatory disease, or sinusitis.

In some embodiments, the disease is an inflammatory disease. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. In some embodiments, the disease is an inflammatory joint disease. In some embodiments, the disease is arthritis. In certain embodiments, the disease is osteoarthritis. In some embodiments, the disease is rheumatoid arthritis.

In some embodiments, the disease is conjunctivitis.

In some embodiments, the disease is aphthous stomatitis.

In some embodiments, the disease is obesity, hyperinsulinemia, or diabetes. In some embodiments, the disease is obesity. In some embodiments, the disease is hyperinsulinemia. In some embodiments, the disease is diabetes. In some embodiments, the disease is type 2 diabetes. In some embodiments, the disease is non-alcoholic fatty liver disease. In some embodiments, the disease is nonalcoholic steatohepatitis.

In some embodiments, the disease is an infectious disease. In some embodiments, the disease is a bacterial, viral, fungal, or parasitic infection. In some embodiments, the disease is a parasitic disease. In some embodiments, the disease is giardiasis, ascariasis, or a tape worm infection. In some embodiments, the disease is schistosomiasis. In some embodiments, the disease is a viral infection. In some embodiments, the disease is influenza. In some embodiments, the disease is SARS-CoV-2 (COVID-19).

In some embodiments, the disease is lactose intolerance.

In certain embodiments, the disease or disorder is a mucosal injury. In some embodiments, the mucosal injury is caused by external agents. In certain embodiments, the mucosal injury is associated with radiation or chemotherapy. In certain embodiments, the mucosal injury is caused by radiation or chemotherapy. In some embodiments, the disease or disorder is mucositis.

In some embodiments, the disorder is a bowel obstruction. In some embodiments, the disorder is a bowel adhesion. In certain embodiments, the methods and compositions described herein prevent re-adhesion and/or re-obstruction of the bowel.

In some embodiments, the disease is trauma.

In some embodiments, the disease is Alzheimer's disease.

In certain embodiments, the polymers and compositions provided herein are contraceptives.

In certain embodiments, the disease is trauma.

In another aspect, the disclosure provides for the use of a composition to form a polymer in vivo comprising administering to a subject a composition comprising a monomer and an oxygen source, wherein the monomer and the oxygen source contact a catalyst endogenous to the subject in vivo and the catalyst polymerizes the monomer, or salt thereof, in situ, and wherein the oxygen source is hydrogen peroxide or urea hydrogen peroxide, and endogenous catalyst is selected from a catalase or a peroxidase. In embodiments, the provided is the use of a composition to form a polymer in vivo comprising administering to a subject a composition comprising a monomer and an oxygen source, wherein the monomer and the oxygen source contact a catalyst endogenous to the subject in vivo and the catalyst polymerizes the monomer, or salt thereof, in situ, and wherein the endogenous catalyst is selected from a catalase or a peroxidase.

In one aspect, the disclosure provides for the use of an effective amount of a composition as described herein to treat a disease or disorder in a subject in need thereof.

In a further aspect, the disclosure provides for the use of an effective amount of a composition as described herein to prevent a disease or disorder in a subject in need thereof.

### Compositions

In certain aspects, further provided herein are compositions comprising a monomer, an oxygen source, optionally a buffer, and optionally, an agent. In some embodiments, the composition further comprises an exogenous catalyst.

In some embodiments, the composition comprises a monomer selected from dopamine, levodopa, norepinephrine, methyldopa, levodopa methyl ester, levodopa ethyl ester, or a monomer comprising: dopamine, levodopa, norepinephrine, methyldopa, levodopa methyl ester, or levodopa ethyl ester; and a macromolecule selected from one or more of alginate, hyaluronic acid, polyacrylic acid, polyethylene glycol, chondroitin sulfate, or chitosan; an oxygen source; optionally, a buffer; and optionally, an agent. In some embodiments, the agent is selected from an active pharmaceutical agent, a cosmetic agent, a nutraceutical agent, an imaging agent, a radioprotective agent, a nutraceutical, an enzyme, an aptamer, an antibody, a neutralizing agent, or a nutrient blocker.

In some embodiments, the composition comprises about 0.001 to about 1000 mg/mL monomer, about 0.01 to about 100 mM of the oxygen source, and optionally, a buffer.

In some embodiments, the composition comprises about 0.001 to about 1000 mg/mL of monomer. In some embodiments, the composition comprises about 0.001 to about 500 mg/mL of monomer. In some embodiments, the composition comprises about 0.01 to about 100 mg/mL of monomer. In some embodiments, the composition comprises about 1 to about 20 mg/mL of monomer. In some embodiments, the composition comprises about 10 mg/mL of monomer. In some embodiments, the composition comprises about 20 mg/mL of monomer.

In some embodiments, the composition comprises 10 mg/mL of a monomer selected from Table 1. In certain embodiments, the composition comprises 10 mg/mL of a monomer selected from Table 1 and 10 mg/mL of a monomer selected from Table 2. In certain embodiments, the composition comprises 10 mg/mL of a monomer selected from Table 1 and 5 mg/mL each of two separate monomers selected from Table 2.

In some embodiments, the composition comprises about 0.01 to about 100 mM of the oxygen source. In some embodiments, the composition comprises about 0.1 to about 50 mM of the oxygen source. In some embodiments, the composition comprises about 1 to about 30 mM of the oxygen source. In some embodiments, the composition comprises about 20 mM of the oxygen source. In some embodiments, the composition comprises a concentration of oxygen source compatible with ingestion by the subject.

In some embodiments, the composition has a pH of about 7 to about 10. In some embodiments, the composition has a pH of about 7 to about 9. In some embodiments, the composition has a pH of about 8.5. In some embodiments, the composition has a pH of about 7.4.

In some embodiments, the composition comprises about 10 mg/mL of monomer, about 20 mM hydrogen peroxide or urea hydrogen peroxide, and optionally, a buffer. In some embodiments, the composition comprises about 20 mg/mL of monomer(s), about 20 mM hydrogen peroxide or urea hydrogen peroxide, and optionally, a buffer.

In some embodiments, the buffer comprises phosphate, acetate, citrate. N-[tris(hydroxymethyl)methyl]glycine), (tris(hydroxymethyl)aminomethane), or (2-(bis(2-hydroxyethyl)amino)acetic acid). In some embodiments, the buffer comprises tris(hydroxymethyl)aminomethane.

In some embodiments, the composition is a liquid or solid dosage form. In some embodiments, the composition is in the form of a solution, a foam, a gel, a tablet, or a capsule, or combinations thereof.

In another aspect, the disclosure provide a method of treating bleeding, comprising administering an effective amount of a composition as described herein to a subject in need thereof. In some embodiments, the bleeding is in the upper GI tract. In certain embodiments, the bleeding is in the stomach. In some embodiments the method of treating bleeding is a method of treating hemostasis.

### Kits

Also encompassed by the disclosure are kits (e.g., pharmaceutical packs). The kits provided may comprise a composition described herein and a container (e.g., a vial, ampule, bottle, syringe, and/or dispenser package, or other suitable container).

The disclosure also provides kits. In one aspect, the disclosure provides a kit comprising: a composition as described herein, and instructions for administering the composition to a subject. In some embodiments, the composition comprises: a monomer as described herein; an oxygen source as described herein; optionally, a buffer as described herein; and optionally, one or more agents as described herein. In some embodiments, the composition comprises: a monomer; an oxygen source; optionally, a buffer; and optionally, one or more agents; and instructions for administering the monomer, and the oxygen source, and optionally the buffer and/or the one or more agents to a subject, such that the monomer and oxygen source contact a catalyst endogenous to the subject in vivo, and wherein the catalyst polymerizes the monomer in situ. In some embodiments, the kit further comprises an exogenous catalyst.

In some embodiments, provided kits may optionally further include a second container comprising a pharmaceutical excipient for dilution or suspension of a composition described herein. In some embodiments, the composition described herein provided in the first container and the second container are combined to form one unit dosage form.

Thus, in one aspect, provided are kits including a first container comprising a composition described herein. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease (in a subject in need thereof. In certain embodiments, the kits and instructions provide for reducing the risk of developing a disease in a subject in need thereof. A kit described herein may include one or more additional agents described herein as a separate composition.

### Administration

The methods and uses described herein comprise administering to a subject an effective amount of a composition comprising a monomer and an oxygen source (i.e., to form a polymer in situ (e.g., in order to treat or prevent a disease).

In some embodiments, the composition is administered orally. In some embodiments, the composition is a liquid or a solid dosage form. In some embodiments, the composition is in the form of a solution, a gel, a foam, a tablet, or a capsule, or combinations thereof.

In certain embodiments, the effective amount is a therapeutically effective amount. In certain embodiments, the effective amount is an amount effective for treating an infectious disease in a subject in need thereof. In certain embodiments, the effective amount is an amount effective for preventing an infectious disease in a subject in need thereof. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., infectious disease, proliferative disease, hematological disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of an organism in a subject or cell.

In certain embodiments, the subject is an animal. The animal may be of either sex and may be at any stage of development. In certain embodiments, the subject described herein is a human. In some embodiments, the subject is an adult human. In certain embodiments, the subject is a child. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Compositions can be prepared, packaged, and/or sold in bulk, as a single unit dose, and/or as a plurality of single unit doses. A “unit dose” is a discrete amount of the composition comprising a predetermined amount of the agent or active ingredient. The amount of the agent or active ingredient is generally equal to the dosage of the agent or active ingredient which would be administered to a subject and/or a convenient fraction of such a dosage, such as one-half or one-third of such a dosage.

Although the descriptions of compositions provided herein are principally directed to compositions which are suitable for administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and/or perform such modification with ordinary experimentation.

Compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific agent or active ingredient employed; the specific composition employed; the age, body weight, general health, sex, and diet of the subject; the time of administration, route of administration, and rate of excretion of the specific agent or active ingredient employed; the duration of the treatment; drugs used in combination or coincidental with the specific agent or active ingredient employed; and like factors well known in the medical arts.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, ophthalmic, intravaginal, intraperitoneal, topical, mucosal, nasal, bucal, sublingual; by intratracheal instillation, bronchial instillation, and/or inhalation; and/or as an oral spray, nasal spray, and/or aerosol. Also, contemplated routes are direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In some embodiments, the route of administration is topical (to skin, eye, ear, mouth, or affected site).

The exact amount of agent or agent or active ingredient required to achieve an effective amount will vary from subject to subject, depending, for example, on species, age, and general condition of a subject, severity of the side effects or disorder, identity of the particular agent or active ingredient, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent or active ingredient described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

A composition, as described herein, can be administered in combination with one or more additional pharmaceutical agents (e.g., therapeutically and/or prophylactically active agents). The compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of an organism in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

The composition can be administered concurrently with, prior to, or subsequent to one or more additional pharmaceutical agents, which may be useful as, e.g., combination therapies.

### Examples

In order that the present disclosure may be more fully understood, the following examples are set forth. The synthetic and biological examples described in this Application are offered to illustrate the compounds, pharmaceutical compositions, methods, and uses provided herein and are not to be construed in any way as limiting their scope.

### Example 1: Evaluation of CAT-Accelerated Polymer Assembly in the Whole Body

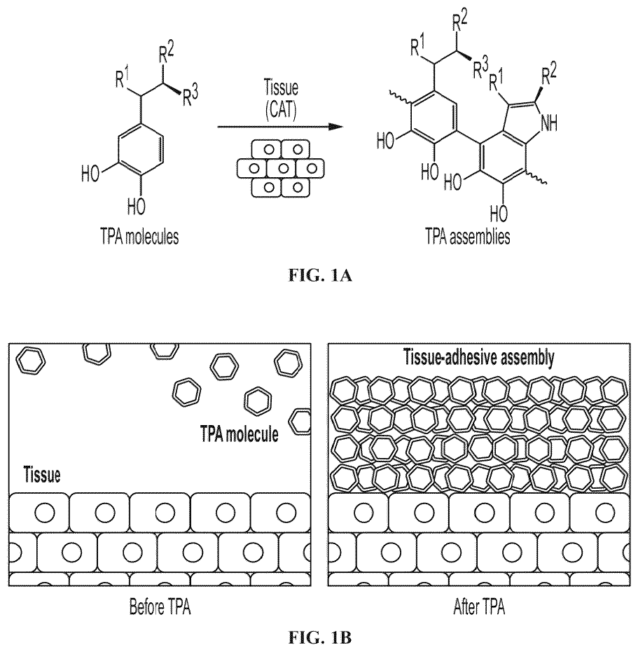

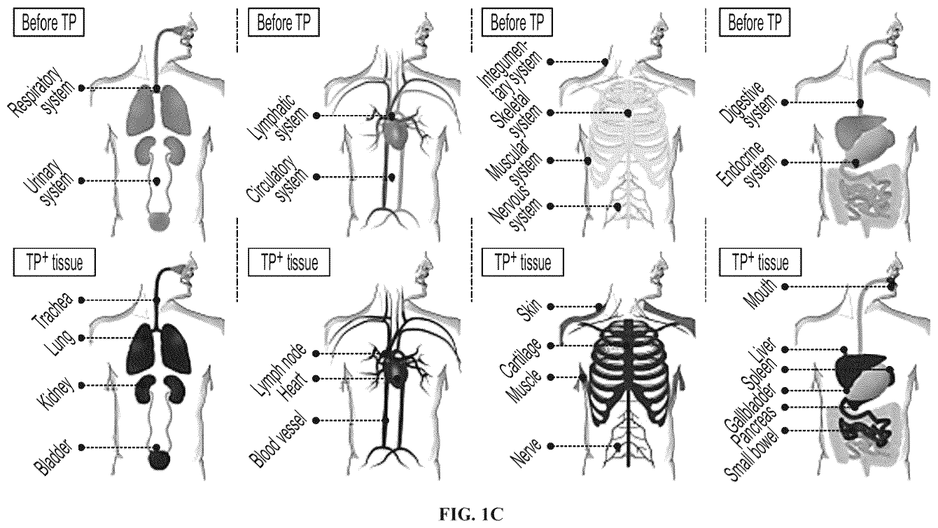

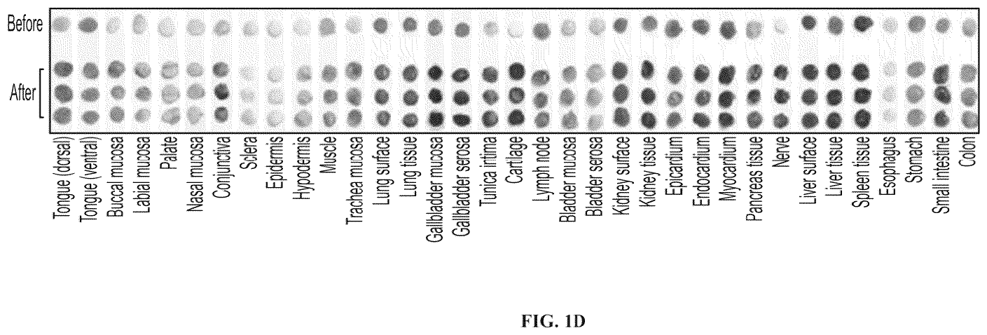

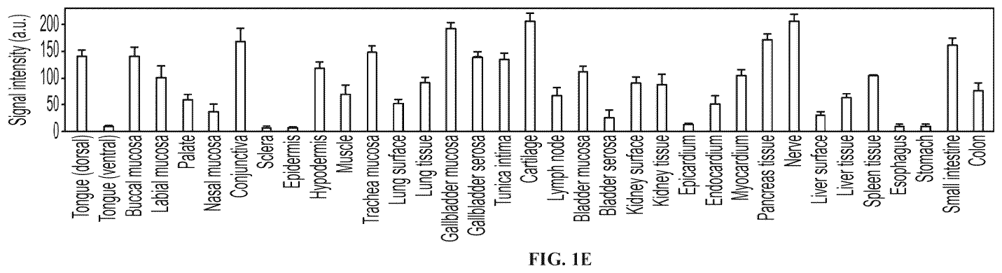

To develop a comprehensive TPA-positive tissue map, fresh tissues from 10 major body systems, including of the respiratory, urinary, lymphatic, circulatory, integumentary, skeletal, muscular, nervous, digestive and endocrine systems (FIG. 1C), were collected and tested for the ability of endogenous catalase (CAT) to expedite dopamine polymerization and coating on the tissue to achieve tissue-accelerated polymerization (TP). This tissue map includes 35 different testing sites across 21 major organs and tissue types, chosen due to their potential as sites for therapeutic and injury interventions. To thoroughly characterize the TPA reactivity of various fresh tissues, porcine tissue was chosen as the first model tissue, due to genomic similarities between humans and pigs. It has been previously shown that human and porcine tissues exhibited similar mRNA and protein expression patterns of CAT^{30,42}. Ex vivo tissues were incubated with the tissue-accelerated polymerization solution, containing dopamine as well as hydrogen peroxide, of which concentrations were within safe oral-consumption levels (0.066% by weight in the solution)⁴³. As shown in FIG. 1C, TPA-positive tissue sites indicate the presence of endogenous CAT capable of catalyzing polymer assembling on top of the tissue surface. CAT is one of the most common enzymes present in the human body, which can break down hydrogen peroxide into oxygen, enabling acceleration of oxygen-driven polymer assembly by multiple orders of magnitude.³⁰ The polymerization of dopamine to polydopamine (PDA), a mussel-inspired tissue adhesive^{44,45}, can be rapidly accelerated upon exposure to hydrogen peroxide and CAT²². Without wishing to be bound by theory, the inventors posit that hydrogen peroxide (either endogenous or supplied as part of the methods or compositions of the present disclosure) diffuses into cells and is rapidly broken down into oxygen by intracellular CAT. Oxygen is then released out of the cells and mixed with extracellular TPA molecules. These TPA molecules near the tissue surface are oxidized into TPA assemblies, which crosslink with biomolecules exposed on the cell surface that are rich in reactive chemical groups like amine, sulfhydryl, and phenol groups. The inventors posit that the instantly crosslinked TPA molecules are of a high molecular weight and undergo crosslinking with the cell surface, which limits their diffusion into the cells.³⁰ Thus, the TPA assembly is a surface-based reaction, expedited by extracellular oxygen generated on the cell surface, that forms a strong polymeric barrier on the tissue. The use of dopamine (colorless) allowed for easy distinction and quantitative characterization of TPA-positive sites (FIG. 1D), as PDA assemblies (dark-brown color) could be characterized by the formation of a dark coloration on the tissue (6 mm in diameter). TPA reactivity was quantitatively evaluated by comparing the signal intensities of tissues before and after PDA assembling (FIG. 1E), and signal increases indicate chromogenic PDA assemblies. Varying levels of PDA assemblies were observed across 35 tissue sites, where 26 tissue sites across 18 major organs (trachea, lung, kidney, bladder, lymph node, heart, blood vessel, eye, skin, cartilage, muscle, nerve, mouth, liver, spleen, gallbladder, pancreas, and small bowel) showed significantly increased PDA levels (P<0.05), but no obvious PDA signal increase was detected in the other 9 tissue sites. These polydopamine coating processes can be visualized due to the chromogenic property of polydopamine. Interestingly, the coating efficiency varies along tissues even within same organs. In the mouth area, polydopamine coating was observed on the tongue (dorsal site), buccal mucosal and labial mucosa, but negligible polydopamine coating was detected on the tongue (ventral site) and palate. In the eye area, polydopamine coating was observed on the conjunctiva but not on the sclera. In the gastrointestinal tract, polydopamine coating was observed on the small intestine but not the stomach and esophagus. These coating differences and specificity are due to different catalase expression levels in different parts of tissues. This extraordinary efficiency and specificity of tissue-accelerated polymerization paves the road for downstream in vivo applications. In addition, within the same organs, different tissue types can perform distinct PDA assembly performance, showing the potential of in vivo targeted medications in these tissues (e.g., tongue, buccal, conjunctiva, skin, trachea, cartilage, bladder, nerve, and small bowel).

### Example 2: An Extensive Library of CAT-Responsive Polymer Assemblies

A combinatorial library of CAT-responsive polymer assemblies was created through three steps: (1) the screening of dopamine analogs to discover additional types of TPA monomers; (2) the conjugation of these TPA monomers onto various polymer backbones to synthesize a series of TPA conjugates and (3) the combination of TPA monomers with TPA conjugates to provide a library of TPA assemblies. The testing solutions contain: 10 mg/ml M1-M6 (one of each), 20 mM H₂O₂, and 50 mM Tris (pH 8.5). The above solution (180 μl for each and total 6 solutions) were prepared fresh and added into 96-well plates, followed by addition of 10 ul catalase (1 mg/ml. The reaction mixture was maintained at 37° C. Extinction of solutions at 700 nm was measured using a plate reader (Tecan). For the controls, the solutions were kept at 37° C. without adding catalase.

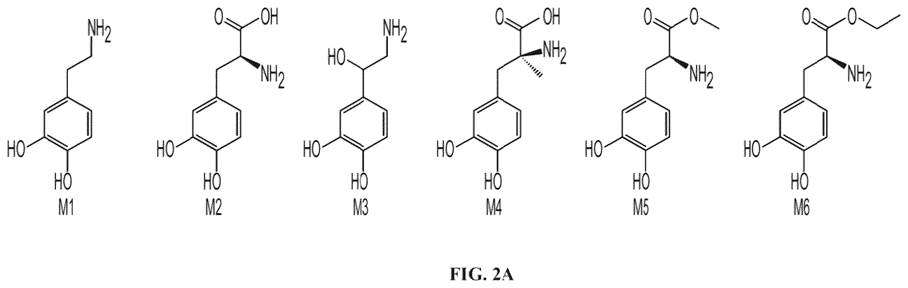

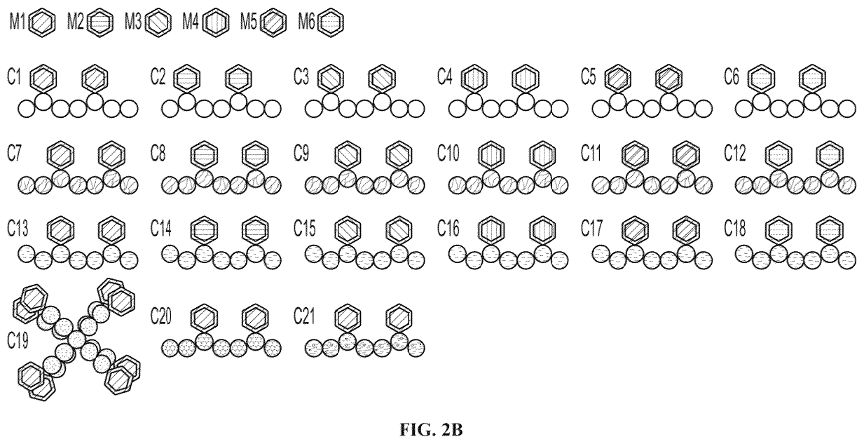

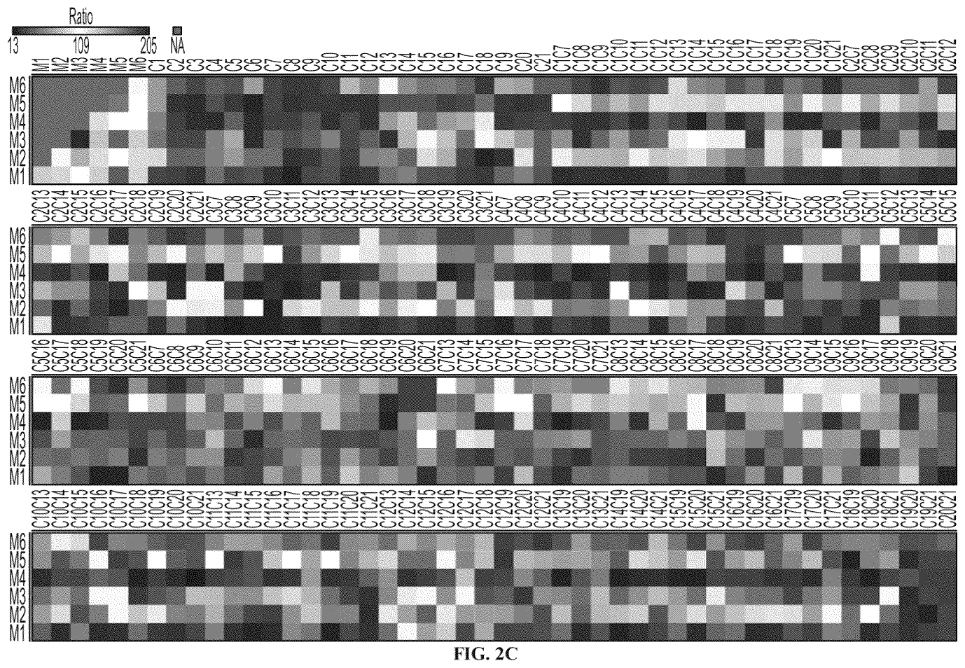

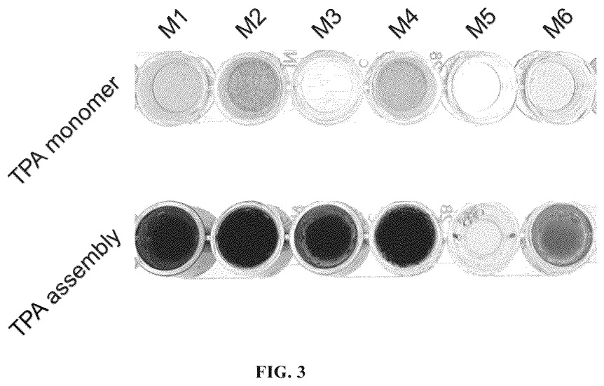

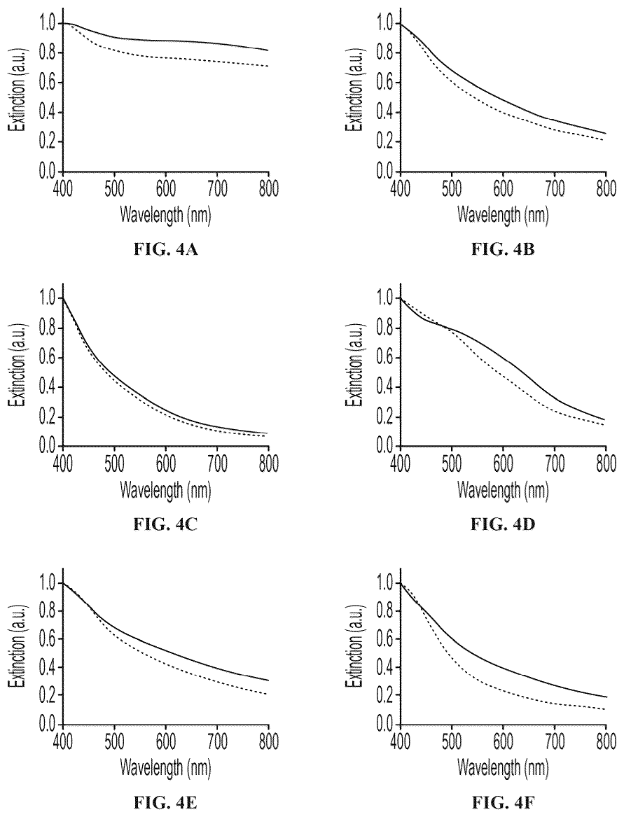

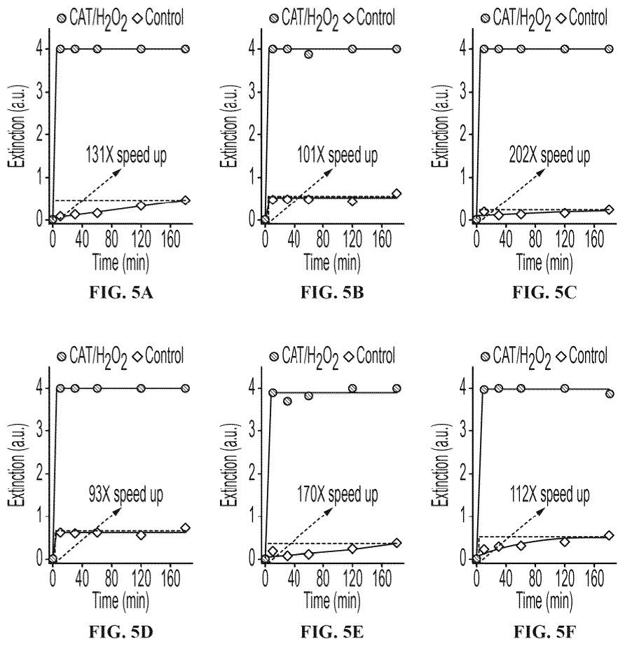

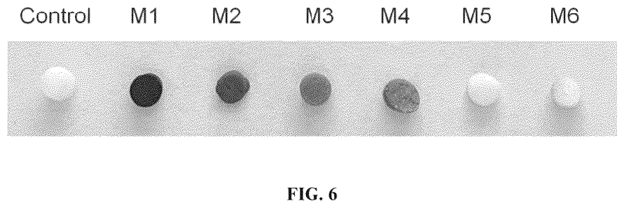

To expand the material combinations of synthetic tissue barriers, monomers other than dopamine were investigated for use in tissue-accelerated polymerization. After screening molecules with similar chemical structures compared to dopamine, five other TPA monomers (M2-M6), including levodopa, norepinephrine, methyldopa, levodopa methyl ester, and levodopa ethyl ester, were discovered as tissue-accelerated polymerization monomers (FIG. 2A). In the screening, the assembly process of dopamine analogs was studied in the presence and absence of commercially purified CAT. The analogs with increased assembly speeds in the presence of CAT signified good candidates for TPA monomers. As shown in FIG. 3, each originally colorless TPA monomer (M1-M6) solution underwent rapid color change upon assembly, allowing visual tracking of assembly progression. It was observed that the color change of the TPA monomer solution (without CAT) is relatively slower as compared to the solution with the addition of CAT, showing that CAT expedites TPA assembly formation. In this study, the structures of TPA assemblies catalyzed by CAT were confirmed to be nearly identical to those of the assembly products without CAT catalysis through ultraviolet-visible (UV-Vis) spectroscopy (FIG. 4). To quantify the effect of catalase on polymerization rates of these tissue-accelerated polymerization monomers (M1-M6), comparisons of reaction kinetics were plotted by measuring the optical extinction of solutions at 500 nm for 3 hours, where all polymeric tissue-accelerated polymerization has a light-absorbing feature. The signals of the catalase-H₂O₂ combination rapidly grew and plateaued within 10 minutes of reactions for all 6 tissue-accelerated polymerization monomers (FIG. 5). However, intensities of light extinction in the conventional condition (in the air) were relatively low, even after 3 hours, of which the same level of intensity was reached within 1-2 minutes under catalase catalysis conditions, showing an increase of polymerization rate by approximately 100-200 times for all TPA monomers. Additionally, it was assessed whether endogenous CAT in tissues could expedite the assembly of these TPA monomers, and subsequently enable adhesion of TPA assemblies to tissues. Ex vivo tissue samples of porcine cartilage were incubated with each TPA monomer solution, and then washed to remove excess unbound TPA assemblies in the solution. As shown in FIG. 6, when white opaque cartilage samples were exposed to TPA monomer solutions, TPA assemblies of corresponding colors were observed on the tissue surface, demonstrating that TPA assemblies were generated in situ in tissues and adhered to biomolecules exposed on the tissue surface. In addition, the color variation of TPA assemblies supplies a tunable optical property to the TPA molecule library and allows the adjustment of barrier appearance for further in vivo applications. Notably, all six identified TPA molecules shared a similar chemical structure of at least one benzene ring with two hydroxyl groups adjacent to one another, an intermediate ethyl chain, and a terminal amine group, classifying the six as catecholamines.

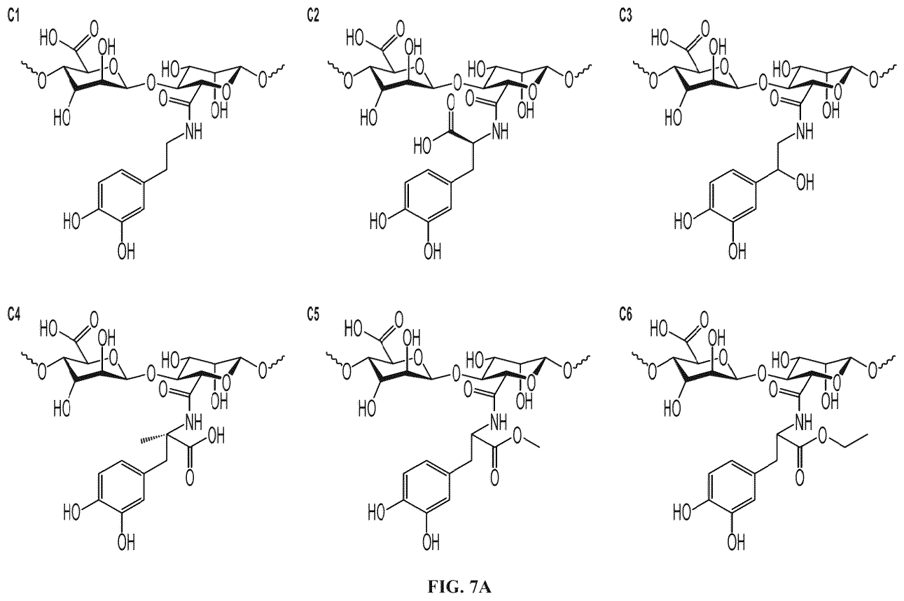

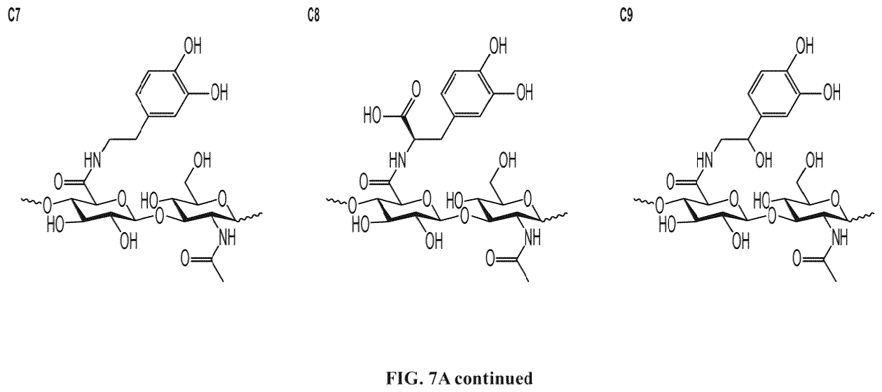

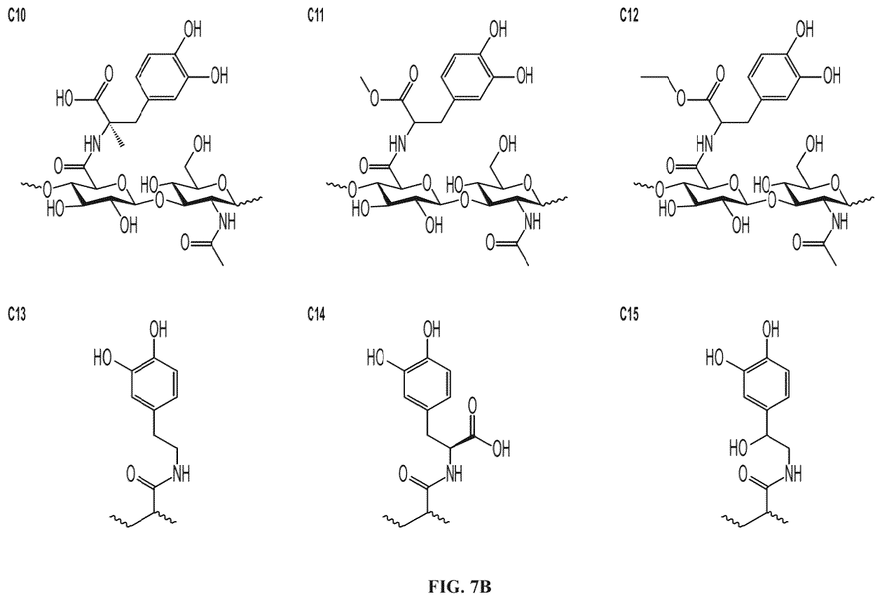

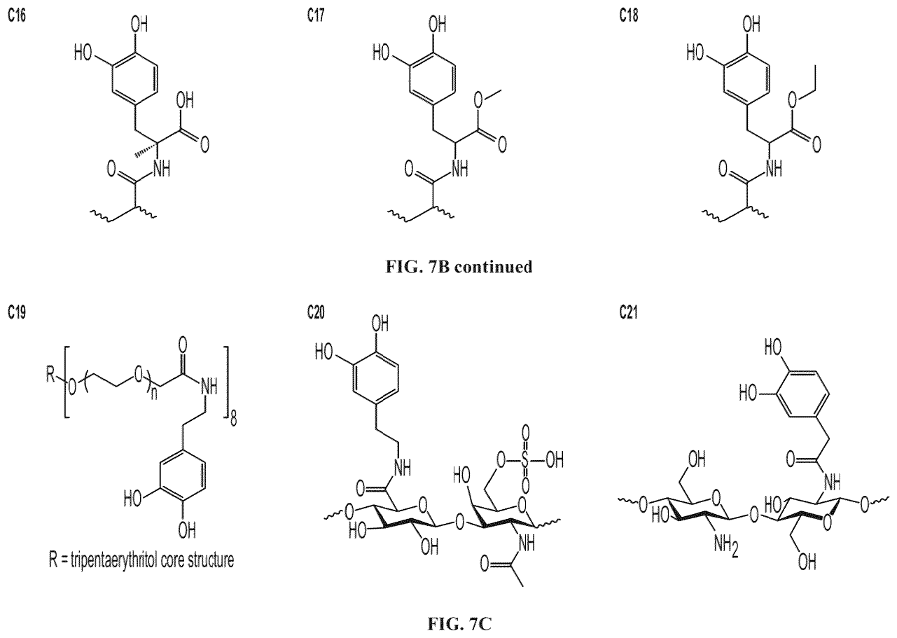

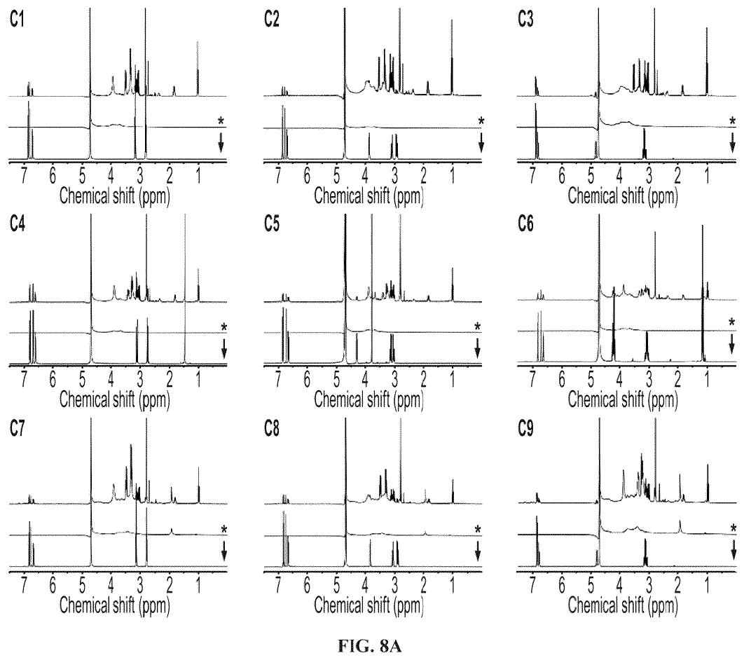

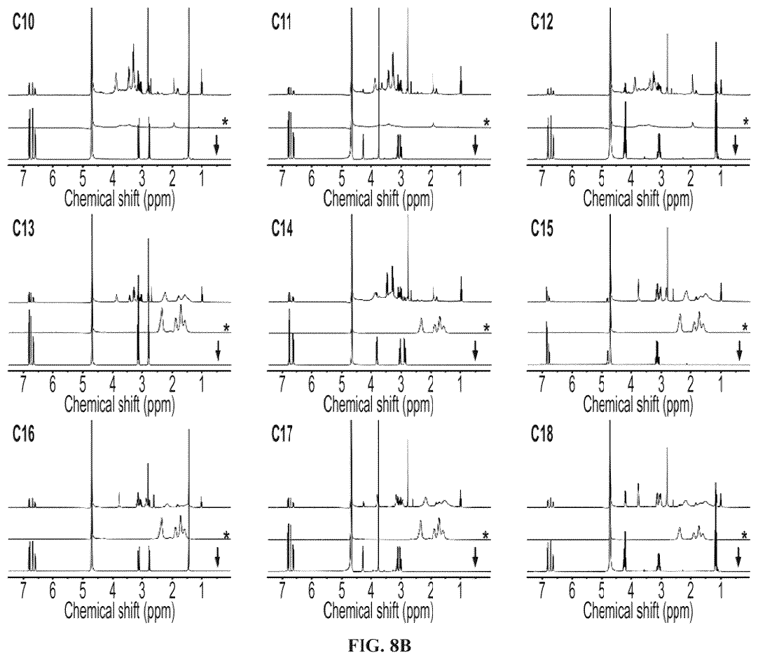

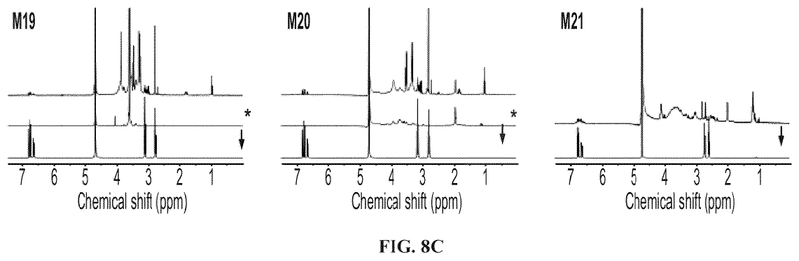

Next, these 6 monomers were conjugated with different biocompatible macromolecules, including alginate, hyaluronic acid, polyacrylic acid, polyethylene glycol, chondroitin sulfate, and chitosan, generating a series of TPA-macromolecule conjugates. After conjugation of TPA components onto the backbones of macromolecules, additional physical and biological functions of these macromolecules could be incorporated into TPA molecules. In this study, six different carboxyl-rich macromolecules were chosen (sodium alginate, hyaluronate, polyacrylic acid, polyethylene glycol, chondroitin sulfate and chitosan) and 21 different TPA-macromolecule conjugates were synthesized (C1-C21; FIG. 7). As shown in FIG. 8, the successful conjugations of these tissue-accelerated polymerization monomers comprising macromolecules were confirmed by using nuclear magnetic resonance (NMR) spectroscopy, where macromolecules, TPA monomers, and conjugated products were separately analyzed and compared for structure similarities and purity.

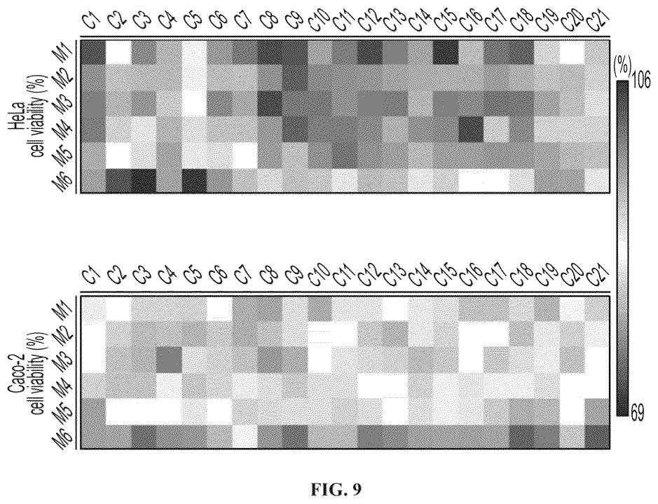

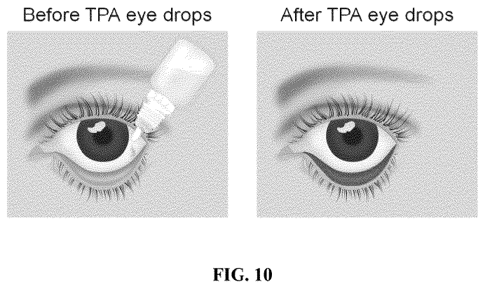

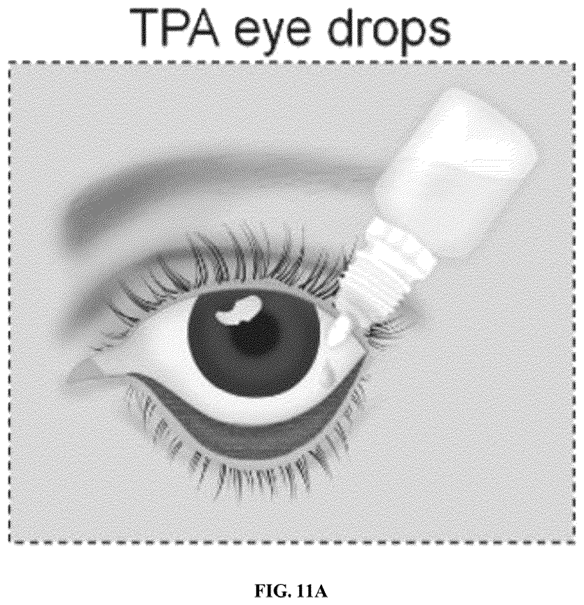

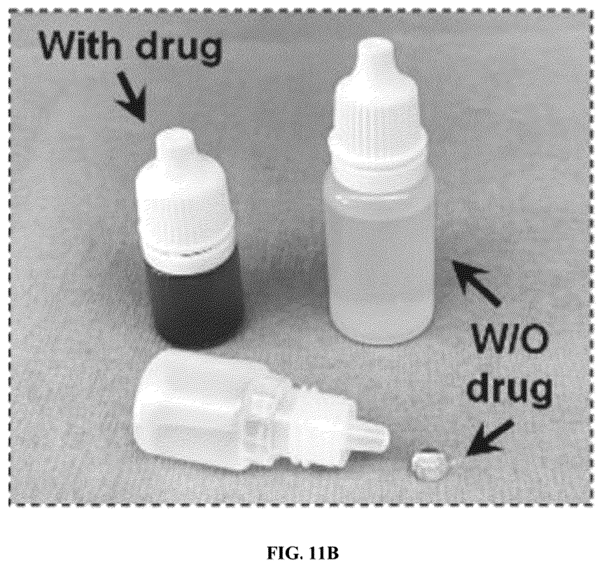

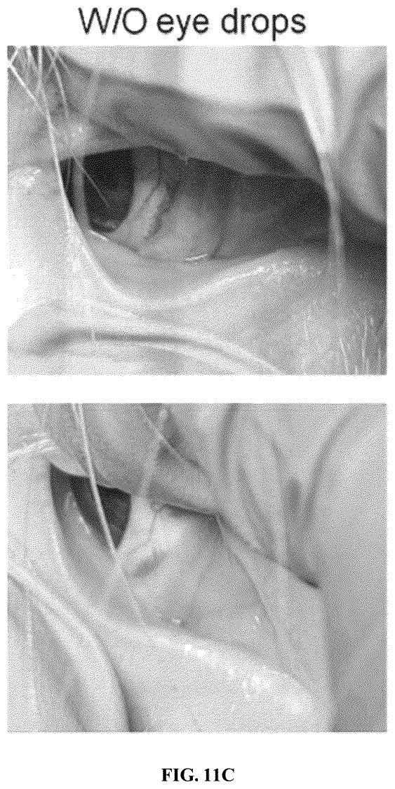

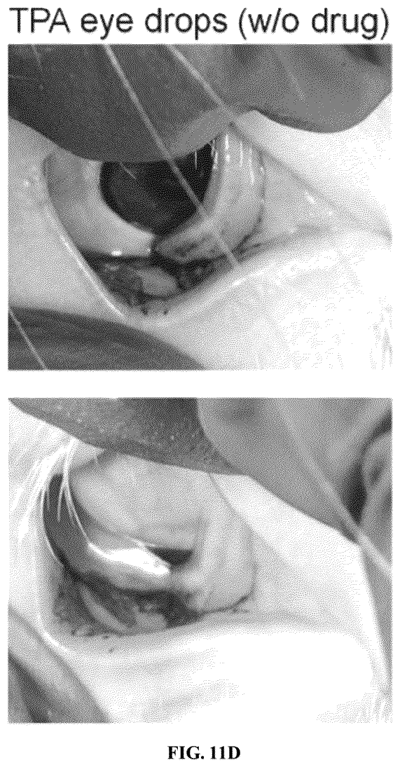

Next, the 6 tissue-accelerated polymerization monomers (M1-M6) and 21 tissue-accelerated polymerization monomers comprising macromolecules (C1-C21) were systematically combined, resulting in crosslinking of multiple polymer networks and the subsequent synthesis of 1,142-membered TPA assemblies. As shown in FIG. 2C, the effect of catalase on polymerization rates was measured for all 1,142 formulations, and shown as the heat map. All formulations are responsive to the catalase with at least 10× to 200× increase of polymerization rates, which demonstrate all materials in the library can be used for tissue-accelerated polymerization and its related potential applications. Also, after adding tissue-accelerated polymerization monomers comprising macromolecules into tissue-accelerated polymerization monomers it was observed that some solutions turn from liquid to gel after tissue-accelerated polymerization, which is due to the crosslinking of macromolecules. Approximately 80% of the tested formulations demonstrated the ability to form a gel due to chemically crosslinked structures in these polymer assemblies, while the remaining formulations remained in the liquid state. The material and formulation tunability offers flexibility such that a particular composition can be tailored for a particular medical use. The results show that M1 is one of the top-performing TPA monomers in terms of gelation capability, and the assembly rates of reactions (M1 with C1-C21) are relatively slower than others (M2-M6 with C1-C21), likely due to limited diffusion of TPA molecules in the rapidly gelled solution with high viscosity. In addition, the biocompatibility of TPA assemblies was evaluated in vitro on HeLa and Caco-2 cell lines (FIG. 9). The cytotoxicity of all typical polymer assemblies on the two cell lines were characterized, and no obvious cytotoxicity was observed after 24-hour in vitro incubation. It was also noted that polymer assemblies generated from C7-C12 showed relatively lower cytotoxicity, which is likely due to the biocompatibility of hyaluronan backbones in C7-C12. Overall, the TPA library offers over 1,000 formulations of CAT-responsive polymer assemblies with various physical and biological properties, holding therapeutic potential for diverse in vivo applications throughout the body.

### Example 3: Tissue-Accelerated Polymerization for Drug Delivery

To evaluate the therapeutic value of the TPA map and TPA library, three different clinical scenarios are exemplified: improvement of administration efficiency for medications in the eye, protection of epithelium against environmental stresses in the oral cavity, and hemostasis treatment of bleeding in the stomach. The TPA map was utilized as a guide for specific tissue-targeting in multiple different tissue sites including conjunctiva, tongue, buccal, and gastric mucosa. Additionally, the TPA library was applied to develop functional barriers across different therapeutic formulations including liquid drops, composite patches, and sprayable powder. In vivo TPA performance was evaluated in a large animal model, Yorkshire pigs (45-75 kg). It is worth noting that pigs were chosen as the model due to their genomic and anatomic similarities to human body systems^{30,46}.

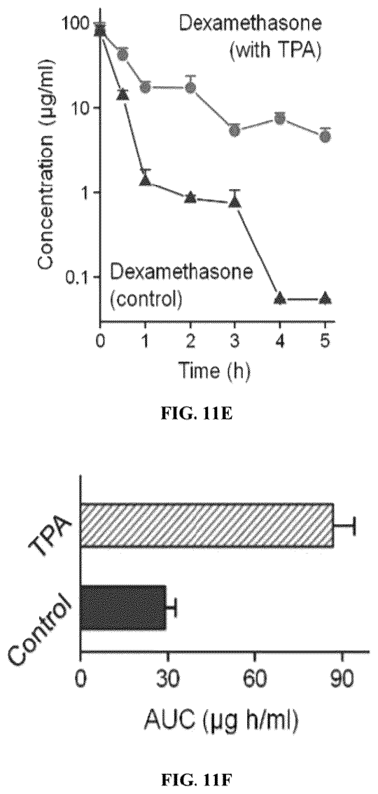

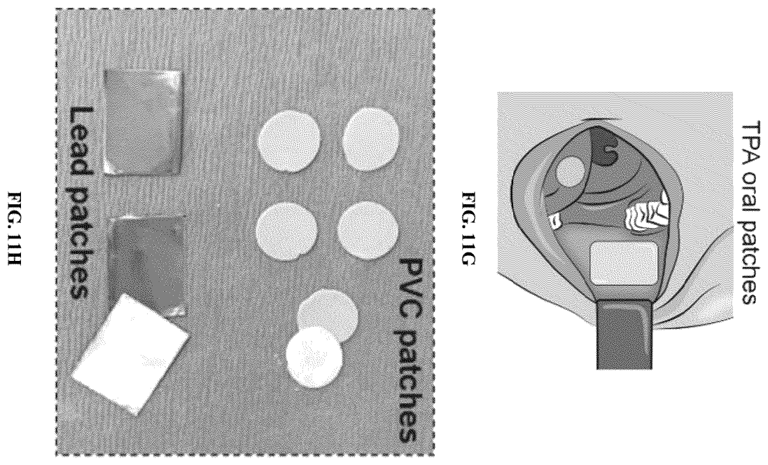

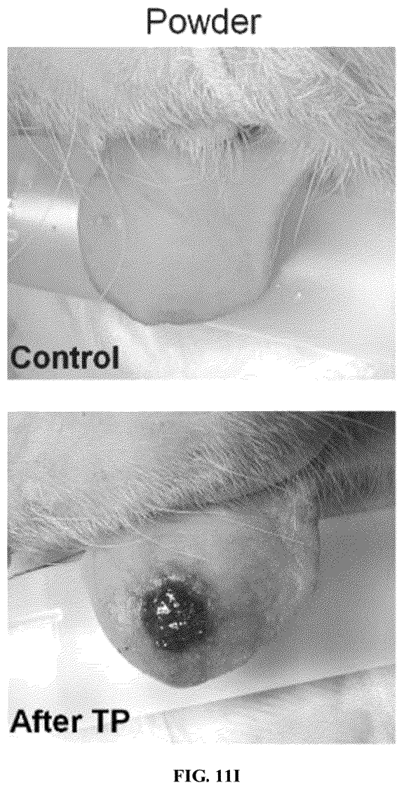

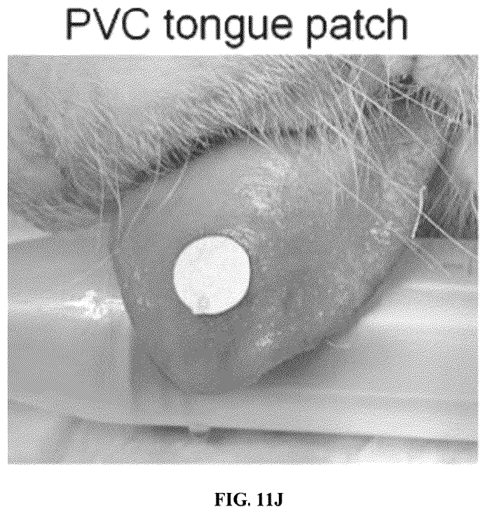

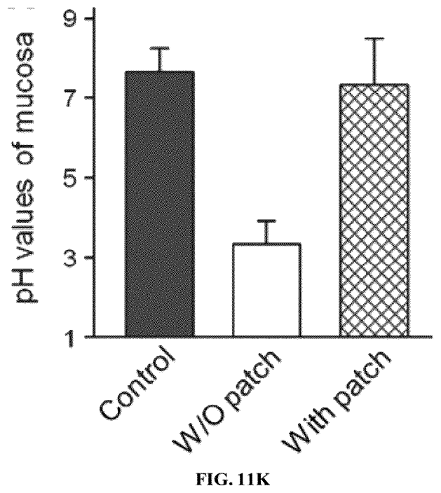

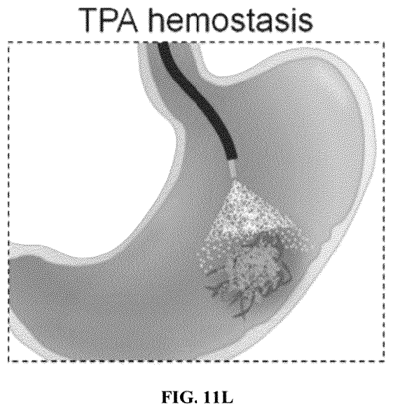

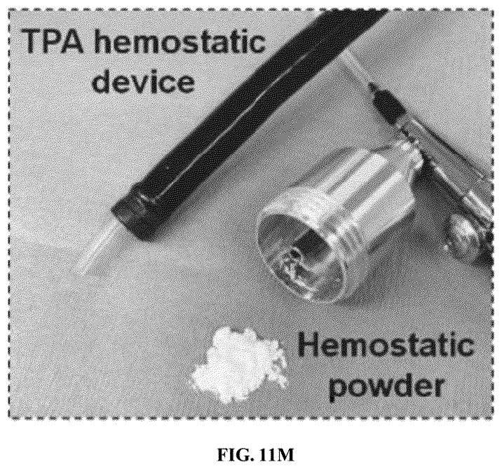

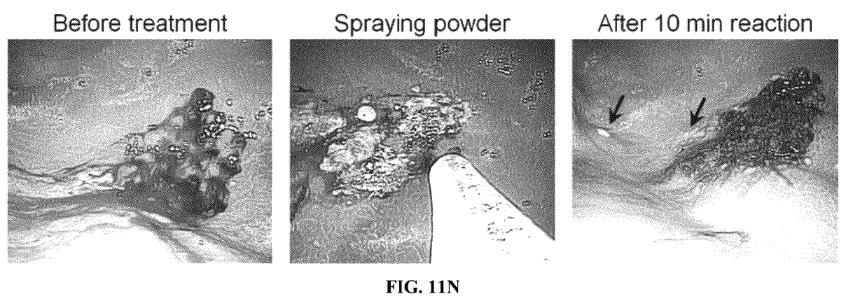

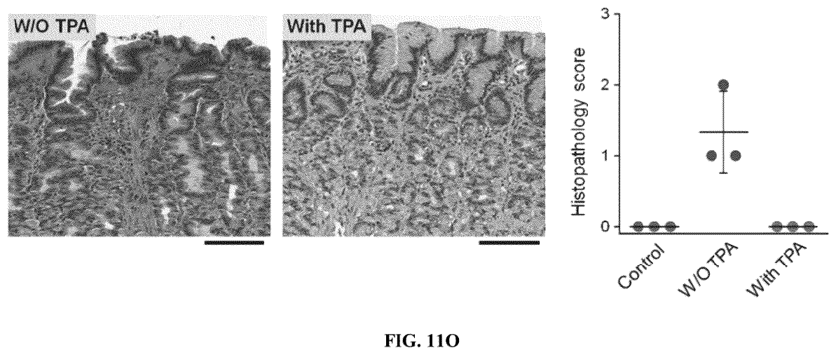

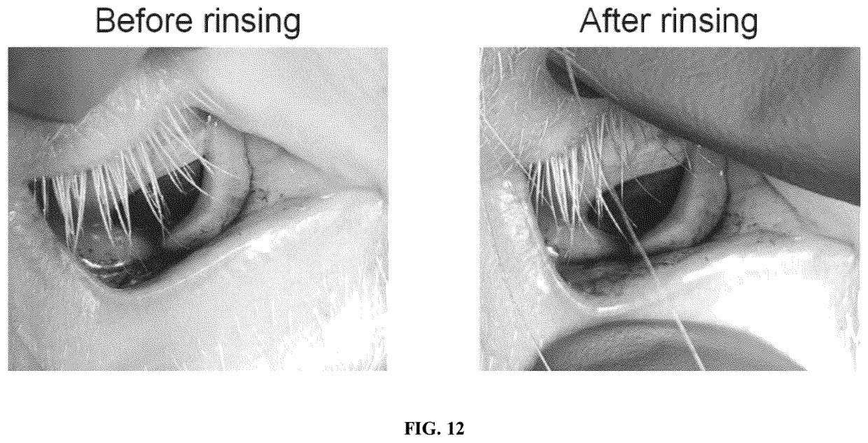

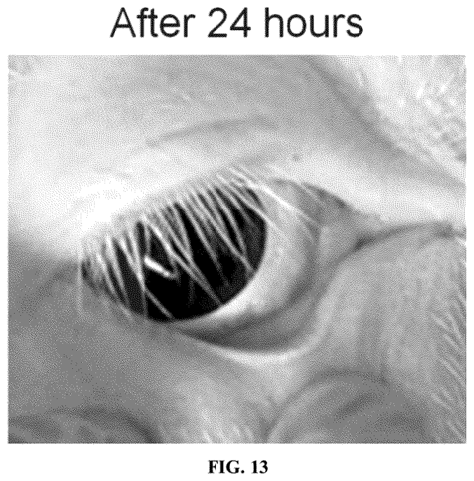

First, the TPA technology was investigated as a system for ocular drug delivery, showing its ability for sustained release of therapeutics in the eye (e.g., for the treatment of conjunctivitis). Conjunctivitis is an ocular disease caused by inflammation or infection of the conjunctiva that affects millions of people annually and imposes huge economic and social burdens.³¹ Corticosteroids, such as dexamethasone, are frequently used to treat conjunctivitis, and are recommended to be frequently administered as eye drops six times per day, which can lead to poor patient adherence and increased risk of glaucoma.³²⁻³³ The development of ophthalmic sustained-release corticosteroids is limited due to the protective mechanism of the eye, which induces drug spillover, tear production, blinking, and nasolacrimal drainage, causing low bioavailability of corticosteroids. The TPA technology disclosed herein can provide a topically applied, conjunctiva-targeted platform for prolonging conjunctival retention and sustained release of drugs in the eye, reducing dosing frequency, and enhancing medication adherence. The TPA map (FIG. 1D) shows efficient polymer assembly in the conjunctiva but not in other parts of the eye, enabling a targeted growth of polymer barriers on the surface of conjunctiva (FIG. 10). To demonstrate the in vivo conjunctiva-targeting ability of TPA in pigs (FIG. 11A), TPA eye drops (FIG. 11B; without drug) containing TPA monomer (M1) were directly administered to eyes of sedated pigs. The eye drops (colorless) were administered into the space created by pulling down the lower eyelid, and the eyelid was released after administration of the eye drops. As shown in FIG. 11C, a dark-brown M1 assembly layer was observed on the surface of the conjunctiva 15 minutes after administration, whereas no such assembly was visualized on the surface of the sclera and cornea, demonstrating targeted M1 assembly and adhesion to the conjunctiva. Additionally, no visualized signal decay was detected after rinsing the conjunctiva with water (FIG. 12), supporting the strong adhesion and stability of assemblies. Notably, these M1 assemblies shed from the eye 24 hours after application (FIG. 13), confirming the transient property of the assemblies. The polymer assemblies, as well as the self-renewing conjunctival epithelium underneath the polymer, are shed together from the eye, which is likely due to the rapid cell turnover and continuous secretion of mucin in the conjunctiva^{47,48}.

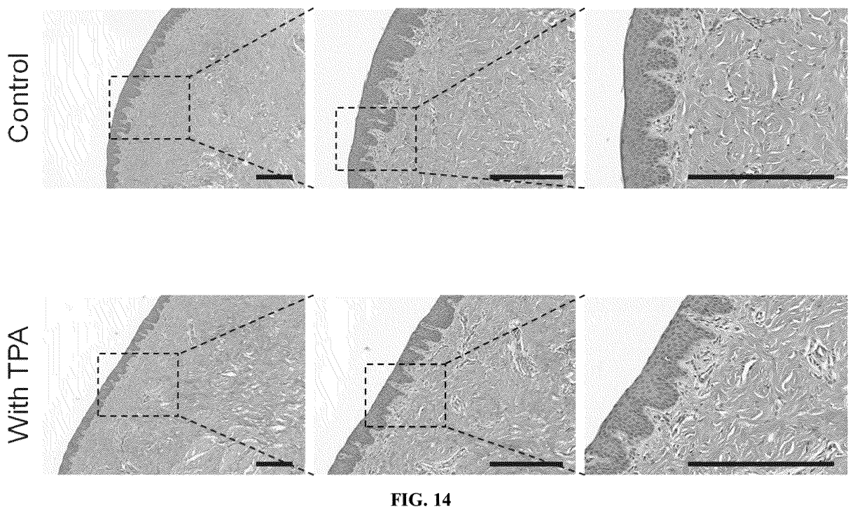

Histological analyses were also performed on the conjunctival epithelium 24 hours after polymer assembly. As shown in FIG. 14, the epithelium remained intact, with staining patterns similar to unexposed controls, supporting the absence of tissue toxicity and the potential safety of TPA eye drops.

Next, dexamethasone was chosen as the model drug to incorporate into TPA eye drops. The surface of the dexamethasone particles was first coated with M1, which enabled chemical crosslinking and incorporation of M1-coated-dexamethasone into polymer assemblies on the conjunctival surface, washed and lyophilized, then resuspended in a solution of trace amounts of H₂O₂ and M1. The solution of coated dexamethasone was dark in color due to the suspension of the TP-coated dexamethasone particles. This coating process enables the drug to crosslink within the TP polymer upon exposure to the CAT on the conjunctiva. Ocular drug release studies were carried out using a single ocular administration of TPA-dexamethasone eye drops in pigs. TPA eyedrops containing suspended M1-coated-dexamethasone particles (dark-black color) were administered to the eyes of pigs in the same manner as described above. After administration, isotonic solution was applied to extract the drug from the conjunctival epithelium at various time points. Drug concentration in the extract was analyzed by liquid chromatography-tandem mass spectroscopy. TPA eye drops (FIG. 11D) and conventional eye drops (without TPA) had comparable initial concentrations of dexamethasone. However, at later times, a higher concentration of dexamethasone was observed when administered using the TPA technology, suggesting a slower rate of drug elimination. This led to a 3-fold increase in the area under the curve (AUC) for the TPA-dexamethasone treated group compared to the conventional dexamethasone treated control group (FIG. 11E), demonstrating sustained drug release. Thus, conjunctiva-targeted TPA eye drops expand the effectiveness of treatment options for conjunctivitis, with potential for other ocular diseases, where medication adherence is essential for therapeutic efficacy.

### Example 4: Tissue-Accelerated Polymerization for Tissue Protection

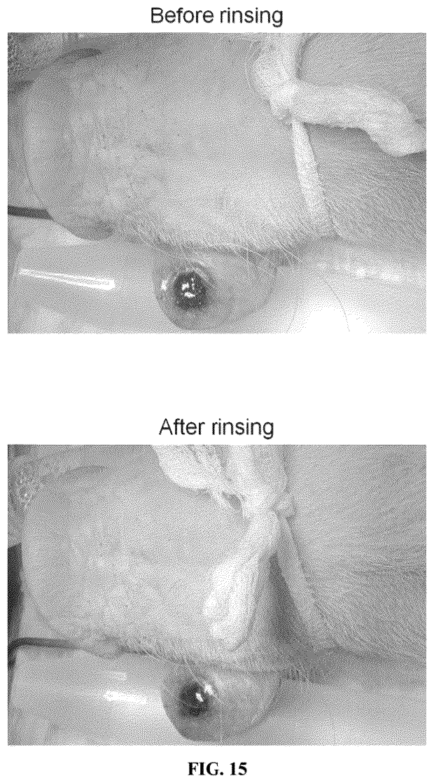

Functional TPA barriers to protect tissues against environmental stresses were developed. The ability of TPA barriers to protect the oral mucosa against chemical and radiation stresses was demonstrated. Oral mucosal diseases, specifically mucositis and aphthous stomatitis, are common ailments that affect numerous people worldwide.³⁴⁻³⁵ Oral mucositis, the inflammation and ulceration of oral mucosa, is an especially common complication among patients who receive radiotherapy and chemotherapy.³⁴ This inadvertent damage to healthy mucosal tissues may lead to treatment discontinuation, and even result in treatment failure and death.³⁶ In addition to mucositis, aphthous stomatitis can also cause painful inflamed ulcers in the oral cavity.³⁵ The treatment of aphthous stomatitis may take several weeks, during which there are many occasions for exacerbated pain, injury, and reinjury due to abrasion or exposure to acidic or spicy foods or drinks. The application of a TP-patch to an ulcer prior to eating could prevent further injury or rupture. In addition, intraoral radiation for the treatment of oral cavity and oropharyngeal cancer can result in inadvertent damage to surrounding healthy tissues. Lead or low-fusing alloy protective splints are widely implemented to prevent damage to adjacent tissues during radiation treatment. Covering mucosal tissues with different protective TPA barriers could enable blocking of both radiation and chemical (acidic) stresses, improving patient prognosis and quality of life. To enable strong tissue-adhesion and flexible protection, TPA barriers were designed as composite patches (FIG. 11G). The patches consist of two layers, including one protective layer made of functional (e.g. radiation shielding and chemical impermeable) materials, and the other tissue-adhesive layer made of TPA molecules (FIG. 1I H). These patches were provided in the form of a flexible sheet, which can be engineered into various shapes and applied onto nonplanar tissue surfaces. Based on the TPA map (FIG. 1D), oral mucosa, such as tongue and buccal mucosa, can expediate TPA and enable assembly deposition on the tissue surface. Thus, the reactivity of the TPA molecule layer in the oral mucosa of pigs was tested first. The dry tissue-adhesive TPA layers (white color), containing TPA monomer (M1) and TPA-macromolecule conjugate (C7), were placed onto the tongues of sedated pigs, which formed dark-brown hydrogel barriers after 15 minutes of administration (FIG. 11I), demonstrating the rapid tissue-directed gelation of TPA molecules on the oral mucosal surface. This gelation is based on the mussel-inspired dry-crosslinking mechanism^{44,49}, where dry TPA compounds can quickly absorb water on wet tissue surfaces upon contact and subsequently strongly crosslink with biomolecules exposed on the tissue surface. In this study, C7, a TPA molecule with relatively lower cytotoxicity (FIG. 9), was incorporated into the formulation, which provided the hydrogel matrix. This hydrogel matrix could effectively dissipate energy under deformation and help the upper protective layer stably adhere to tissues. To test the tissue adhesion stability of the hydrogel, the tongue surface was vigorously rinsed with the water (FIG. 15), during which the hydrogel remained strongly attached on the surface of the tongue.

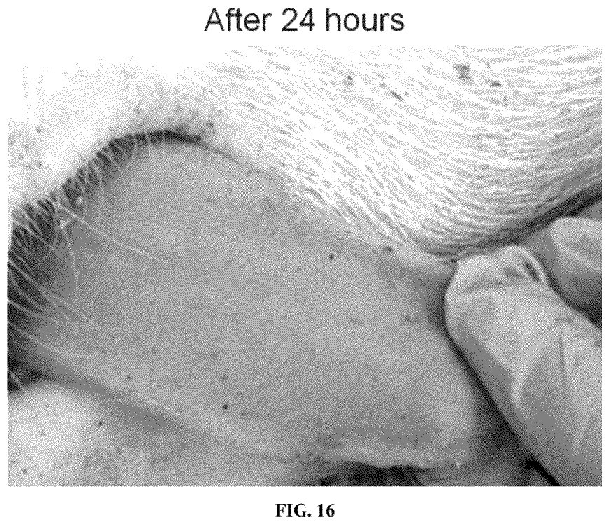

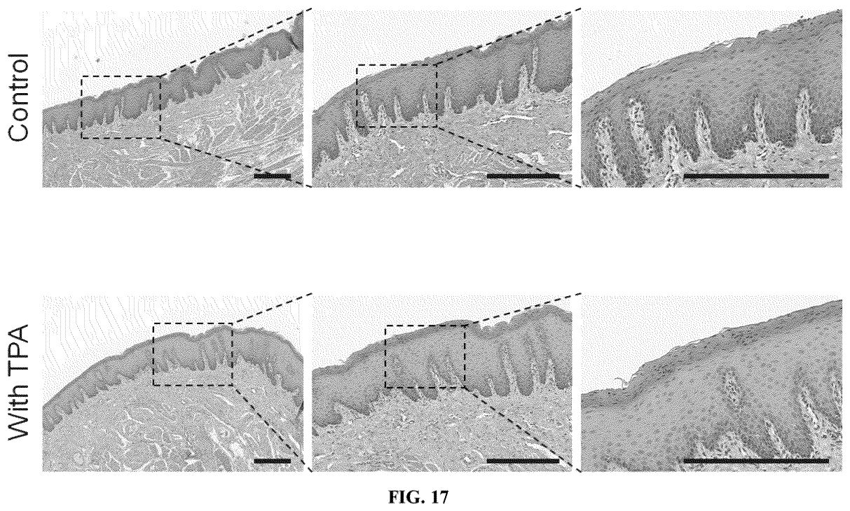

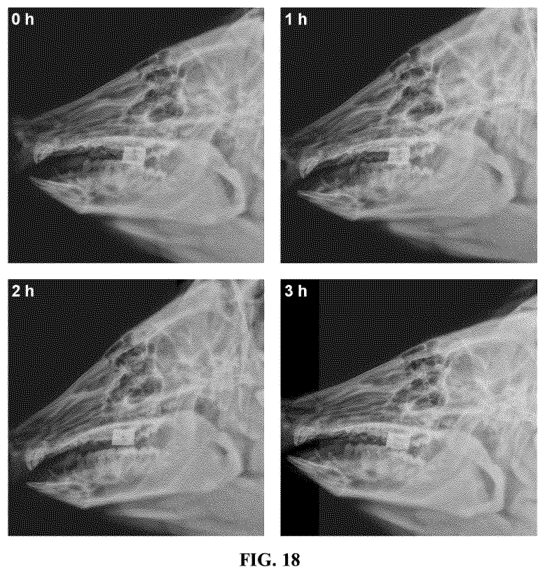

Additionally, the hydrogel shed from the tongue 24 hours after application (FIG. 16), confirming its transient property, which is likely due to the fast turnover of oral epithelium through rapid stem cell proliferation⁵⁰. The absence of tissue toxicity was confirmed through histological analyses of tissues 24 hours after hydrogel formation (FIG. 17), showing the potential safety of TPA oral patches. Second, two different protective layers, a lead sheet and a thin polyvinyl chloride (PVC) foam, were attached on the back of tissue-adhesive TPA layers separately. Lead, with a high atomic number, is capable of attenuating radiation dose exposure, and is widely implemented to protect healthy tissues during radiation diagnosis and treatment. In this study, TPA-lead patches were placed onto the buccal surface of sedated pigs, and a series of X-ray images of the same anatomic location were taken periodically. As shown in FIG. 11J and FIG. 18, the TPA-lead patches remained strongly attached on the buccal mucosa for 3 hours, sufficient for the time-length (within minutes) of typical radiotherapy⁵¹.

A variation of the TP-patch was designed for testing with pH paper interfacing with the tissue on the tongue. Upon application of acid to the tongue without the TP-patch, the pH drops to an average of 3. When the TP-patch is adhered and acid applied, the pH remains at levels comparable to that of the control normal pH of the tongue. The resilience of pH levels in the presence of acid with the TP-patch demonstrate the protective capabilities of the TP-patch for resistance against acidic foods that could further irritate ulcers in the mouth.

Next, tissue-adhesion and chemical permeability of the TPA-PVC patches were tested. To validate the blocking function against chemicals, specifically acid, TPA-PVC patches were placed onto the surface of the tongues of sedated pigs, citric acid solution (pH 3) was applied on top of the patches after 15 minutes of application, and the pH of the mucosa underneath the patches was measured to quantify the acid blocking efficiency. As shown in FIG. 11K, control pigs, without TPA-PVC patches, showed dramatically decreased mucosal pH after the citric acid solution application. However, pH values of mucosa treated with TPA-PVC patches remained at levels comparable to healthy control values. This resistance to pH changes demonstrated the protective capability of TPA-PVC patches against acid or chemical penetration, which is likely due to the hydrophobicity of the PVC foam layer, supporting the applications of these patches for protecting ulcers against acidic stresses that could exacerbate pain and injury in the oral cavity. These results address the clinical need for protective patches in the oral cavity, supporting the flexibility of TPA technology for potentially shielding various environmental stresses through the incorporation of different blocking materials.

### Example 5: Tissue-Accelerated Polymerization for Hemostasis Treatment

TPA technology was used to treat upper gastrointestinal bleeding by generating a TPA barrier specifically over the bleeding site in the stomach (FIG. 11L). Gastrointestinal bleeding is one of the most urgent emergencies in a clinic that represents the most frequent cause of hospitalizations.³⁷ Ineffective hemostasis of upper gastrointestinal bleeding, mostly due to peptic ulcers, can result in high-volume blood loss, risk of perforation, and even complications or death.³S The rate of mortality in patients with gastrointestinal bleeding is around 10%^{[0001]7}. The most common treatment for acute upper gastrointestinal bleeding is endoscopic hemostasis, such as epinephrine injection, thermal coagulation, mechanical hemostasis using hemoclips, and noncontact modalities.^{[001]8,39-40} However, these procedures require expertise on quickly recognizing the bleeding point, which often causes re-bleeding and limits the therapeutic efficacy.⁴¹ In addition, these approaches can be challenging, especially for bleeding sites that are hard to access. The present disclosure provides an alternative technology capable of specifically targeting the bleeding point to efficiently control bleeding and simplify the existing endoscopic hemostasis. In particular, a tissue-accelerated polymerization based hemostasis method is disclosed that does not require direct contact with the bleeding point, preventing the inducement of further damage or more severe bleeding. Without wishing to be bound by theory, the inventors posit that the strong catalase expression in blood cells would result in gel formation once in contact with tissue-accelerated polymerization powders according to the present disclosure. The targeting of bleeding points was achieved due to high CAT activity of blood relative to the stomach^{52,53}. The stomach is a TPA-negative site based on the TPA map (FIG. 1D), and the healthy gastric mucosa could not expedite TPA barrier formation and adhesion on the tissue surface. However, the blood from the bleeding peptic ulcer could prompt TPA barrier formation, which would cover over the bleeding point and subsequently achieve hemostasis. To demonstrate the catalytic capacity of the blood for TPA reactions, fresh porcine blood was collected through tubes containing sodium citrate anti-coagulant, and then mixed with the TPA hemostatic powder (M1 and C7 as TPA molecules). Here M1C7 was used as the formulation for proof of concept and used as a powder comprising M1 (500 mg), C7 (50 mg), Tris or sodium carbonate or potassium carbonate powder (60 mg), solid urea H₂O₂ (50 mg). The powder was mixed and sprayed onto the bleeding site through endoscopic catheter powered by the compressed air. This demonstrated that M1C7 together with other excipients can induce blood gelation and coagulation to address gastrointestinal bleeding.

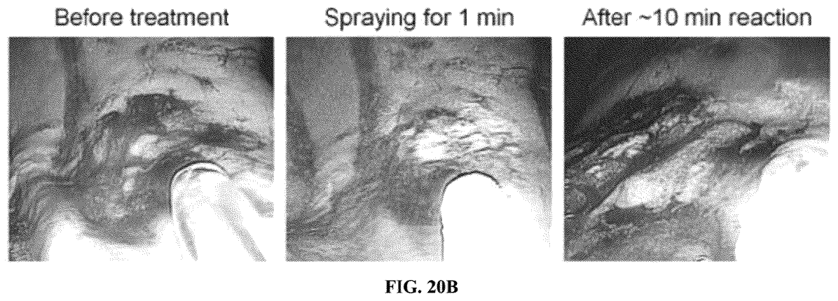

Blood coagulation in Yorkshire pigs in vivo was then tested in a stomach bleeding model. The advantages of using pigs as the large animal model include their extensive homology with the human genome, anatomical similarity to the human gastrointestinal tract, and physiological likeness to the human digestive system. Gastrointestinal endoscopy together with forceps were used to induce the bleeding in the stomach to mimic the acute upper gastrointestinal bleeding. After inducing bleeding, tissue-accelerated polymerization powder (T1 (500 mg), M7 (50 mg), Tris or sodium carbonate or potassium carbonate powder (60 mg), solid urea H₂O₂ (50 mg)) was continuously sprayed to the bleeding sites through a delivery catheter that was inserted inside the working channel of the endoscopy. The end of the spray device was connected with compressed air setup to propel the tissue-accelerated polymerization powder out of the catheter. Gastrointestinal endoscopy was used for real-time recording of blood gelation and coagulation after spraying the tissue-accelerated polymerization powder. As shown in FIG. 20, a dark-brown tissue-accelerated polymerization gel was observed on the bleeding sites instantly after tissue-accelerated polymerization powder spraying, whereas no such tissue-accelerated polymerization gel was visualized in areas without bleeding or with the control powder after the same spraying. The bleeding site was efficiently covered by tissue-accelerated polymerization gels that formed a barrier over vessel walls and stopped the bleeding. The tissue-accelerated polymerization barrier formation was specific targeted towards the blood, and had no effect on healthy tissue in the stomach. Compared to conventional hemostatic powders, this specific targeting property has the potential to reduce side effects induced by the non-specific tissue adhesion of the powders. Also, tissue-accelerated polymerization powder can help tissue recovery and regeneration, which was demonstrate through both endoscopy imaging and histology (FIGS. 9 and 10).

### Example 6: Further Example of Tissue-Accelerated Polymerization for Hemostasis Treatment

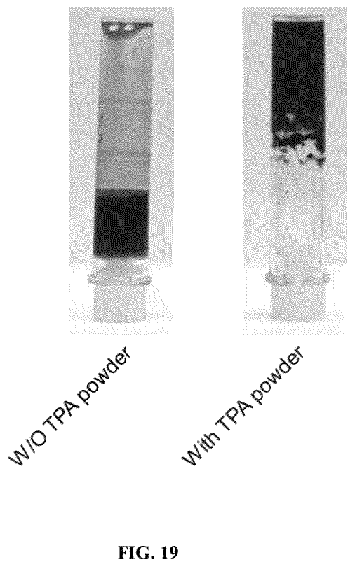

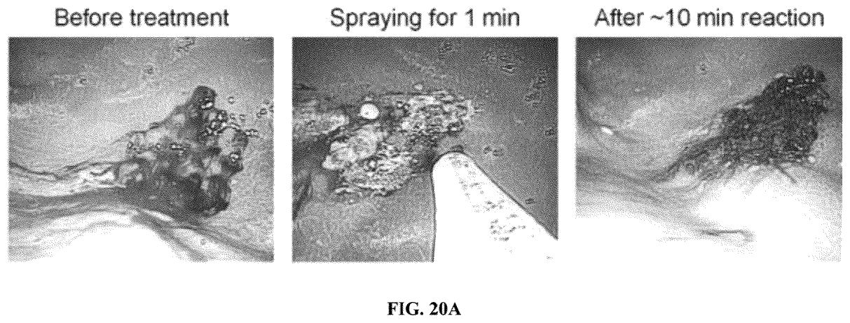

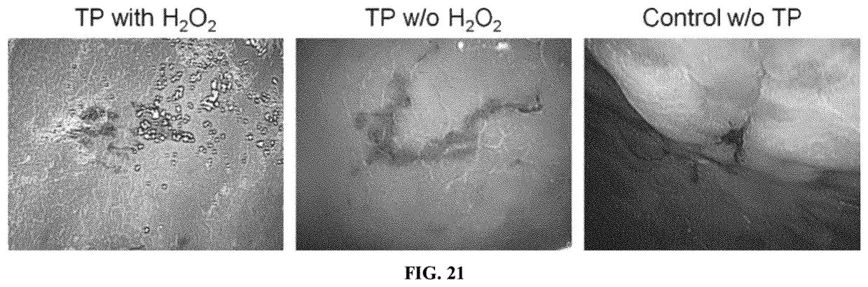

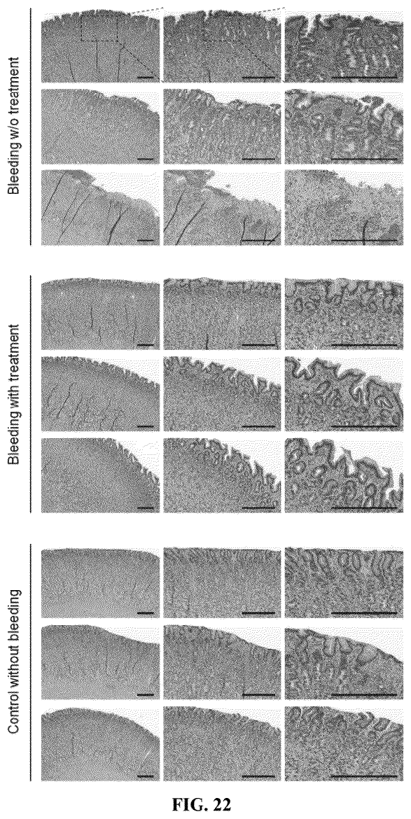

As shown in FIG. 19, the blood (red liquid) instantly turned to a black gel once mixed with the TPA powder, demonstrating the accelerated polymer assembling and crosslinking of TPA molecules under blood catalysis. To further evaluate the hemostatic capacity of the TPA technology, an acute stomach-bleeding (Forrest IB bleeding) pig model was used to test the safety and effectiveness of endoscopically applied TPA hemostatic powder in vivo⁵⁴. Under moderate sedation, endoscopic biopsy forceps were utilized to generate a 10-20 mm puncture wound (without perforation) in the stomach, resulting in bleeding (oozing hemorrhage) from the puncture site. Under endoscopic visual guidance, the TPA hemostatic powder was then sprayed toward the bleeding site by using a TPA hemostatic device (FIG. 11M), which consists of a delivery catheter inserted through the working channel of the endoscope and a hemostatic powder container connected to compressed air, propelling the hemostatic powder out of the delivery catheter. Gastrointestinal endoscopy videography was used for the recording of the treatment process. Within 10 minutes of the hemostatic powder (white color) application, a black gel barrier was observed to form on the bleeding site (FIG. 11N), where the bleeding was effectively stopped due to expediated blood clotting. However, no such barrier formation was visualized when the control powder (without TPA gelation ingredients) was applied (FIG. 20B), and the bleeding could not be controlled in time. Notably, during the delivery of TPA hemostatic powder, some powders were also inevitably deployed on the healthy tissue around the bleeding site (FIG. 11O), whereas these powders remained unreacted in the light-yellow/white color, demonstrating the specific targeting of TPA technology to the bleeding point but not healthy tissue. These results support the hemostatic capacity of the endoscopic TPA hemostasis, and that its effectiveness is likely due to the instant adhesion of the powder upon contact with the wet tissue surface, the increased concentration of clotting factors due to blood dehydration, the rapid blood-directed gelation of TPA molecules on the targeted bleeding site, and the strong adherence of the gel-barrier due to chemical crosslinking between TPA molecules, blood, and the gastric epithelium. In addition, no powder or gel-barrier was endoscopically identified in the stomach 24 hours after hemostasis treatment (FIG. 21), showing that all hemostatic materials were eliminated from the stomach due to rapid epithelial restitution⁵⁵. Additionally, a non-flat ulceration was endoscopically visualized at the puncture site of the stomach in animals without TPA hemostasis treatment (FIG. 21), a result of conventional wound healing⁵⁶. However, when TPA hemostasis was applied, a flat and almost completely healed wound was observed through endoscopic examination, showing that the TPA gel-barrier also promoted wound healing. The mechanism underlying the accelerated healing of gastric mucosa is likely due to two factors, including the physical protection provided from the TPA gel-barrier against gastric environmental stresses, and the physiological function of hyaluronan backbones in TPA molecules that can improve wound healing⁵⁷. In this study, it was also observed that control powder (hyaluronan-based TPA molecules alone) could help with the wound healing (FIG. 21) but was less efficient relative to the TPA hemostatic powder. To further confirm the therapeutic efficacy of the TPA hemostasis at cellular and tissue levels, gastric tissues at the wound site were harvested for histological examination 24 hours after the hemostasis treatment. Consistent with the endoscopic results, microscopic and histological analyses of formalin-fixed paraffin-embedded (FFPE) tissue specimens revealed apparent epithelium regeneration of damaged gastric mucosa exposed to the TPA hemostatic powder, whereas incomplete regenerative epithelium and loss of mucosal integrity were observed in unexposed controls (FIG. 11O and FIG. 22). Furthermore, these histological images were scored to evaluate the severity of the mucosal damage in different conditions⁵⁸ (FIG. 11P), showing a significant difference between TPA hemostasis treated groups and groups without treatment (P<0.05). However, the scoring of mucosal integrity is not significantly different between TPA treated groups and healthy control groups (P>0.05), demonstrating the successful epithelial restitution after TPA treatment. No hemostatic powder or gel barrier was histologically identified in all tissue samples (FIG. 22), showing that all TPA materials were not absorbed by the mucosal tissue, indicative of a minimal risk for systemic toxicity for future human studies. Collectively, these results reveal that the TPA hemostatic powder promoted wound healing by providing a protective, transient, and therapeutic barrier over the wound site. Therefore, the TPA technology opens an avenue to specifically target bleeding points in the stomach, and a promising mechanism to control gastrointestinal bleeding and promote lesion healing.

### Example 7: Tissue-Accelerated Polymerization for Material Retention in Eye Conjunctiva, Cartilage, and Oral Cavity for Potential Sustained Drug Release

To demonstrate the versatility of the tissue-accelerated polymerization platform, the material retention of tissue-accelerated polymerization formulation in eye conjunctiva, cartilage, and oral cavity was evaluated.

A solution formulation was prepared for the eye comprising 10 mg/ml M1 and 20 mM H₂O₂, dissolved in 50 mM Tris buffer (pH 8.5). The solution (1 ml) was applied onto the conjunctiva for 15 minutes and then washed with water.

A solution formulation was prepared for the cartilage comprising 10 mg/ml M1 and 20 mM H₂O₂ dissolved in 50 mM Tris buffer (pH 8.5). The solution (3 ml) was intra-articular injected into the cartilage through needles. After 15 minutes, the cartilage was taken out and washed with water for evaluation.

A powder formulation was prepared for the oral cavity comprising M1 (500 mg), M7 (50 mg), Tris or sodium carbonate or potassium carbonate powder (60 mg), solid urea H₂O₂ (50 mg). The powder was sprayed on top of the tongue or buccal and after 15 minutes, the tissue was washed with water for evaluation. In some embodiments, different drugs are incorporated into the system to treat eye disease (e.g. glaucoma) by eye drop formulation, to treat joint diseases (e.g. osteoarthritis) as intra-articular injection formulation, to protect oral mucosa from damage as barrier coatings or drug patches. In some embodiments, drugs are incorporated as a powder, particle, oil, or dissolved liquid forms and are co-delivered together with the tissue-accelerated polymerization formulation. After in-situ polymerization and crosslinking, these drugs are trapped into the polymer, that enable slow and sustained release. In embodiments, the drug is timolol, latanoprost, or bimatoprost and are used to treat the eye. In certain embodiments, the drug is an interleukin 1 receptor antagonist, insulin-like growth factor 1, dexamethasone, or triamcinolone and are used for intra-articular injection. In some embodiments, the drug is nystatin, clotrimazole, fluconazole, or dexamethasone and are used for delivery to the oral cavity.

### Example 8: Evaluation of TPA in Human Tissues

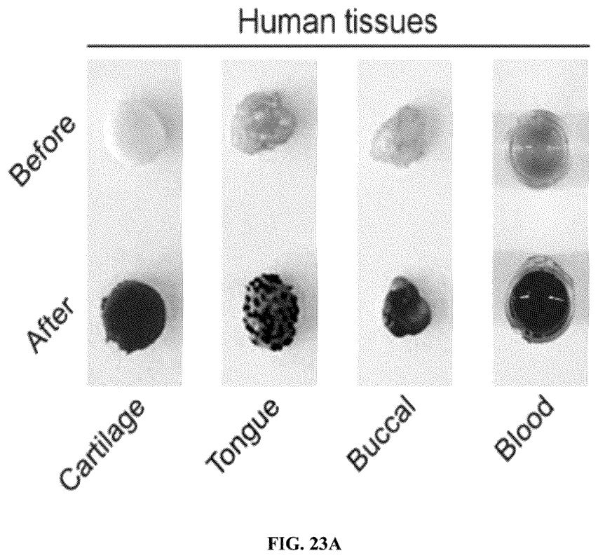

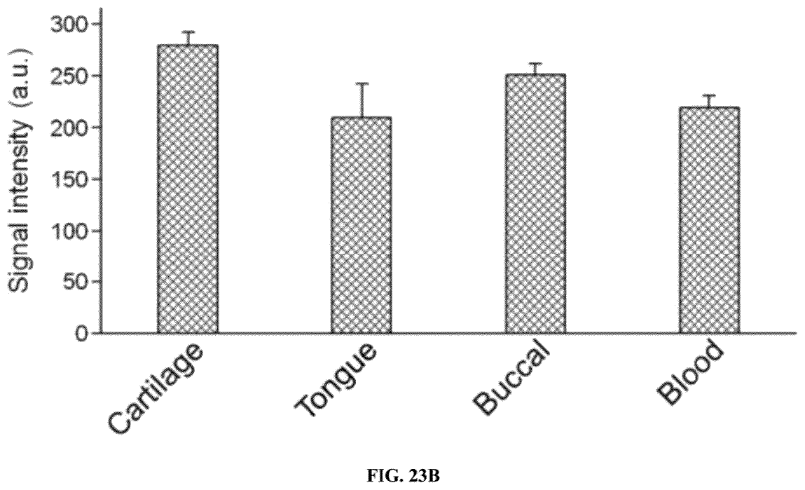

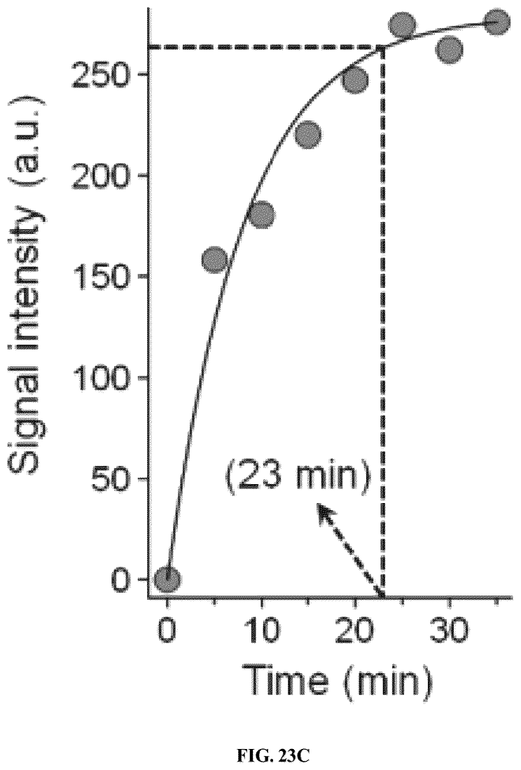

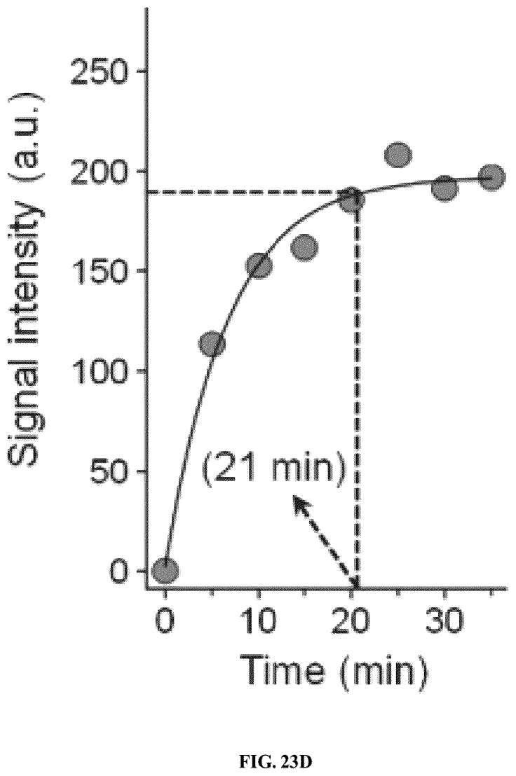

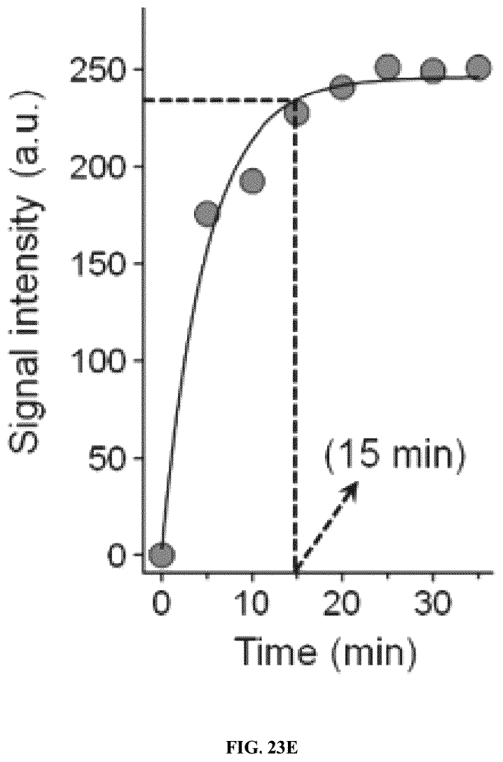

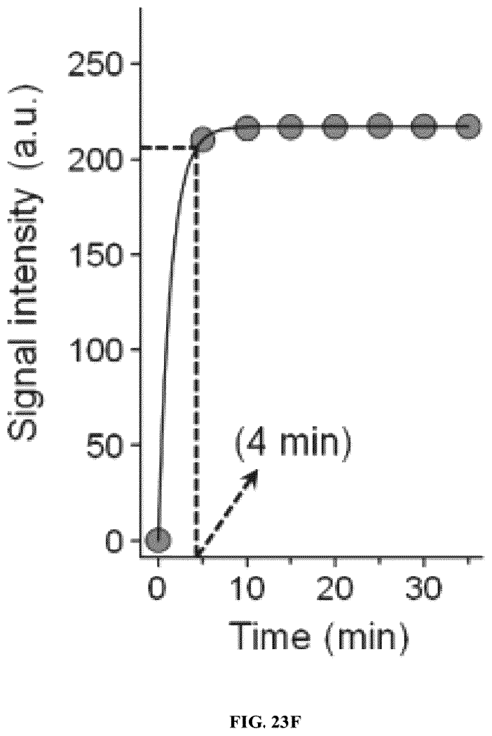

TPA technology has been developed to be applicable to human tissues and suitable for clinical translation. To confirm the reliability of the porcine-based TPA-positive tissue map described above for potential human trials, fresh resected tissue specimens from humans were tested for compatibility with TPA technology. Similar to the porcine TPA-map study, human tissue specimens were incubated with the TPA solution containing M1, and subsequently CAT-accelerated chromogenic M1 assembly was characterized. As shown in FIG. 23A, stably adhered M1 assemblies were observed across various human tissues, including cartilage, buccal, tongue, and blood. These TPA barriers on tissue surfaces were additionally confirmed through quantitative signal analysis (FIG. 23B), and the signal intensities of M1 assemblies were highly consistent with porcine results. Additionally, the M1 assembly formation kinetics showed rapid signal developments for all tissue types, where 95% completion was reached within ˜4-23 minutes (FIG. 23C-F), supporting rapid TPA barrier growth in human tissues. These results revealed high correlation of TPA activity between porcine and human tissues, which is likely due to their similar CAT distribution in the body and ultimately their genomic similarities^{30,42,53}. Thus, the porcine-based TPA map, that covers 35 different testing sites across 21 major organs and tissue types, can serve as a potential guide for future medication developments in various human tissue sites and forthcoming preclinical and clinical trials.

### REFERENCES

1. Anselmo, A. C., Gokarn, Y. & Mitragotri, S. Non-invasive delivery strategies for biologics. Nat. Rev. Drug Discov. 18, 19-40 (2018).
2. Zelikin, A. N., Ehrhardt, C. & Healy, A. M. Materials and methods for delivery of biological drugs. Nat. Chem. 8, 997-1007 (2016).
3. Lee, Y. et al. Therapeutic luminal coating of the intestine. Nat. Mater. 17, 834-842 (2018).
4. Yui, S. et al. Functional engraftment of colon epithelium expanded in vitro from a single adult Lgr5+ stem cell. Nat. Med. 18, 618-623 (2012).
5. Kitano, K. et al. Bioengineering of functional human induced pluripotent stem cell-derived intestinal grafts. Nat. Commun. 8, 765 (2017).
6. Elloumi-Hannachi, I., Yamato, M. & Okano, T. Cell sheet engineering: a unique nanotechnology for scaffold-free tissue reconstruction with clinical applications in regenerative medicine. J. Intern. Med. 267, 54-70 (2010).
7. Nahon, S. et al. Epidemiological and prognostic factors involved in upper gastrointestinal bleeding: Results of a French prospective multicenter study. Endoscopy 44, 998-1006(2012).
8. Vanleerdam, M. et al. Acute upper GI bleeding: did anything change? Am. J. Gastroenterol. 98, 1494-1499 (2003).
9. Candi, E., Schmidt, R. & Melino, G. The cornified envelope: A model of cell death in the skin. Nat. Rev. Mol. Cell Biol. 6, 328-340 (2005).
10. Lambrecht, B. N. & Hammad, H. The airway epithelium in asthma. Nat. Med. 18, 684-692 (2012).
11. Obermeier, B., Daneman, R. & Ransohoff, R. M. Development, maintenance and disruption of the blood-brain barrier. Nature Medicine vol. 19 1584-1596 (2013).
12. Peterson, L. W. & Artis, D. Intestinal epithelial cells: regulators of barrier function and immune homeostasis. Nat. Rev. Immunol. 14, 141-153 (2014).
13. Zihni, C., Mills, C., Matter, K. & Balda, M. S. Tight junctions: From simple barriers to multifunctional molecular gates. Nat. Rev. Mol. Cell Biol. 17, 564-580 (2016).
14. Varga, Z. et at. Endothelial cell infection and endotheliitis in COVID-19. Lancet 395, 1417-1418 (2020).
15. Richard, M. et at. Influenza A viruses are transmitted via the air from the nasal respiratory epithelium of ferrets. Nat. Commun. 11, 1-11 (2020).
16. Nishida, K. et at. Corneal reconstruction with tissue-engineered cell sheets composed of autologous oral mucosal epithelium. N. Engl. J. Med. 351, 1187-1196 (2004).
17. Gallico, G. G., O'Connor, N. E., Compton, C. C., Kehinde, O. & Green, H. Permanent coverage of large burn wounds with autologous cultured human epithelium. N. Engl. J. Med. 311, 448-451 (1984).
18. Torres, J., Mehandru, S., Colombel, J. F. & Peyrin-Biroulet. L. Crohn's disease. Lancet 389, 1741-1755 (2017).
19. Sweeney, M. D., Sagare, A. P. & Zlokovic, B. V. Blood-brain barrier breakdown in Alzheimer disease and other neurodegenerative disorders. Nat. Rev. Neurol. 14, 133-150 (2018).
20. Sun, Y. et al. Inhibition of autophagy ameliorates acute lung injury caused by avian influenza A H5N1 infection. Sci. Signal. 5, ra16-ra16 (2012).
21. Lee, Y. et al. Therapeutic luminal coating of the intestine. Nat. Mater. 17, 834-842 (2018).
22. Vacanti, J. P. & Langer, R. Tissue engineering: The design and fabrication of living replacement devices for surgical reconstruction and transplantation. Lancet 354, 32-34 (1999).
23. Anselmo, A. C., Gokarn, Y. & Mitragoui, S. Non-invasive delivery strategies for biologics. Nature Reviews Drug Discovery vol. 18 19-40 (2018).
24. Mohanaruban, A. et al. PTH-003 Endobarrier®: a safe and effective novel treatment for obesity and type 2 diabetes? Endoscopy 66, A205.1-A205 (2017).
25. Khademhosseini, A. & Langer, R. A decade of progress in tissue engineering. Nat. Protoc. 11, 1775-1781 (2016).
26. Carino, G. P. & Mathiowitz, E. Oral insulin delivery. Adv. Drug Deliv. Rev. 35, 249-257 (1999).
27. Freedman, B. R. & Mooney, D. J. Biomaterials to mimic and heal connective tissues. Adv. Mater. 31, (2019).
28. Taboada, G. M. et al. Overcoming the translational barriers of tissue adhesives. Nat. Rev. Mater. 5, 310-329 (2020).
29. Odenwald, M. A. & Turner, J. R. The intestinal epithelial barrier: a therapeutic target?Nat. Rev. Gastroenterol. Hepatol. 14, 9-21 (2017).
30. Li, J. et al. Gastrointestinal synthetic epithelial linings. Sci. Transl. Med. 12, 441 (2020).
31. Azari, A. A. & Barney, N. P. Conjunctivitis: A systematic review of diagnosis and treatment. JAMA—Journal of the American Medical Association vol. 310 1721-1729 (2013).
32. FDA. MAXIDEX-dexamethasone ophthalmic suspension Label. FDA, US. (2019).
33. Kersey, J. P. & Broadway, D. C. Corticosteroid-induced glaucoma: A review of the literature. Eye vol. 20 407-416 (2006).
34. Sonis, S. T. The pathobiology of mucositis. Nat. Rev. Cancer 4, 277-284 (2004).
35. Scully, C. Aphthous Ulceration. N. Engl. J. Med. 355, 165-172 (2006).
36. Vera-Llonch, M., Oster, G., Hagiwara, M. & Sonis, S. Oral mucositis in patients undergoing radiation treatment for head and neck carcinoma: Risk factors and clinical consequences. Cancer 106, 329-336 (2006).
37. Lanas, A. et al. Non-variceal upper gastrointestinal bleeding. Nat. Rev. Dis. Prim. 4, 18020 (2018).
38. Laine. L. Upper Gastrointestinal Bleeding Due to a Peptic Ulcer. N. Engl. J. Med. 374, 2367-2376 (2016).
39. Gralnek, I. M., Barkun, A. N. & Bardou, M. Management of acute bleeding from a peptic ulcer. N. Engl. J. Med. 359, 928-937 (2008).
40. Barkun, A. N., Moosavi, S. & Martel, M. Topical hemostatic agents: A systematic review with particular emphasis on endoscopic application in GI bleeding. Gastrointest. Endosc. 77, 692-700 (2013).
41. Chahal, D., Lee, J. G. H., Ali-Mohamad, N. & Donnellan, F. High rate of re-bleeding after application of Hemospray for upper and lower gastrointestinal bleeds. Dig. Liver Dis. 52, 768-772 (2020).
42. Godin, D. V. & Garnett, M. E. Species-related variations in tissue antioxidant status-I. Differences in antioxidant enzyme profiles. Comp. Biochem. Physiol.—Part B Biochem. Mol. Biol. 103, 737-742 (1992).
43. U.S. Food and Drug Administration. Oral health care drug products for over-the-counter human use; antigingivitis/antiplaque drug products; establishment of a monograph; proposed rules. Fed. Regist. 68, 32232-32287 (2003).
44. Maier, G. P., Rapp, M. V., Waite, J. H., Israelachvili, J. N. & Butler, A. Adaptive synergy between catechol and lysine promotes wet adhesion by surface salt displacement. Science (80-.). 349, 628-632 (2015).
45. Lee, H., Dellatore, S. M., Miller, W. M. & Messersmith, P. B. Mussel-inspired surface chemistry for multifunctional coatings. Science 318, 426-30 (2007).
46. Traverso, G. & Langer, R. Perspective: special delivery for the gut. Nature 519, S19-S19 (2015).
47. Lavker, R. M. & Sun, T. T. Epithelial stem cells: The eye provides a vision. Eye 17, 937-942 (2003).
48. Gipson, I. K. Goblet cells of the conjunctiva: A review of recent findings. Progress in Retinal and Eye Research vol. 54 49-63 (2016).
49. Yuk, H. et al. Dry double-sided tape for adhesion of wet tissues and devices. Nature (2019) doi:10.1038/s41586-019-1710-5.
50. Squier, C. & Brogden, K. A. Human Oral Mucosa: Development, Structure and Function. John Wiley & Sons (2010).
51. Gerber, D. E. & Chan, T. A. Recent Advances in Radiation Therapy. Am. Fam. Physician 78, 1254-1262 (2008).
52. Goyal, M. M. & Basak, A. Human catalase: Looking for complete identity. Protein Cell 1, 888-897 (2010).
53. Fagerberg, L. et al. Analysis of the human tissue-specific expression by genome-wide integration of transcriptomics and antibody-based proteomics. Mol. Cell. Proteomics 13, 397 (2014).
54. Forrest, J. A. H., Finlayson, N. D. C. & Shearman, D. J. C. Endoscopy in gastrointestinal bleeding. Lancet 304, 394-397 (1974).
55. Sáenz, J. B. & Mills, J. C. Acid and the basis for cellular plasticity and reprogramming in gastric repair and cancer. Nat. Rev. Gastroenterol. Hepatol. 15, 257-273 (2018).
56. Arakawa, T. et al. Quality of ulcer healing in gastrointestinal tract: Its pathophysiology and clinical relevance. World J. Gastroenterol. 18, 4811-4822 (2012).
57. Xu, X. et al. Bioadhesive hydrogels demonstrating pH-independent and ultrafast gelation promote gastric ulcer healing in pigs. Sci. Transl. Med 12, (2020).
58. ANDREWS, F. M. et al. Comparison of endoscopic, necropsy and histology scoring of equine gastric ulcers. Equine Vet. J. 34, 475-478 (2010).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-165. (canceled)

166. A solid dosage form comprising an active pharmaceutical agent, a monomer, and an oxygen source, wherein:
the monomer comprises dopamine and a macromolecule, wherein the macromolecule comprises alginate, hyaluronic acid, polyacrylic acid, polyethylene glycol, chondroitin sulfate, chitosan, or a combination thereof; and
when administered to a subject, the oxygen source contacts a catalyst in a tissue of the subject, and release of oxygen from the oxygen source polymerizes the monomer on the tissue; and
the active pharmaceutical agent is retained or encapsulated in the polymer.

167. The solid dosage form of claim 166, wherein the monomer comprises dopamine and alginate.

168. The solid dosage form of claim 166, wherein the oxygen source is hydrogen peroxide or urea hydrogen peroxide.

169. The solid dosage form of claim 166, wherein the catalyst is a catalase or a peroxidase.

170. The solid dosage form of claim 166, wherein the active pharmaceutical agent is a diagnostic agent, contraceptive, statin, anorexic, anti-hypertensive, antibiotic, antiparasitic, anti-proliferative agent, anti-cancer agent, anti-angiogenesis agent, anti-inflammatory agent, immunosuppressant, anti-bacterial agent, anti-viral agent, cholesterol-lowering agent, anti-diabetic agent, anti-allergy agent, antiparkinsonism drug, or pain-relieving agent.

171. The solid dosage form of claim 170, wherein the active pharmaceutical agent is an anorexic or anti-diabetic agent.

172. The solid dosage form of claim 170, wherein the active pharmaceutical agent is an antibiotic.

173. The solid dosage form of claim 172, wherein the antibiotic is amoxicillin.

174. The solid dosage form of claim 170, wherein the active pharmaceutical agent is an antiparkinsonism drug.

175. The solid dosage form of claim 166, wherein the solid dosage form is a tablet.

176. The solid dosage form of claim 166, wherein the monomer comprises:

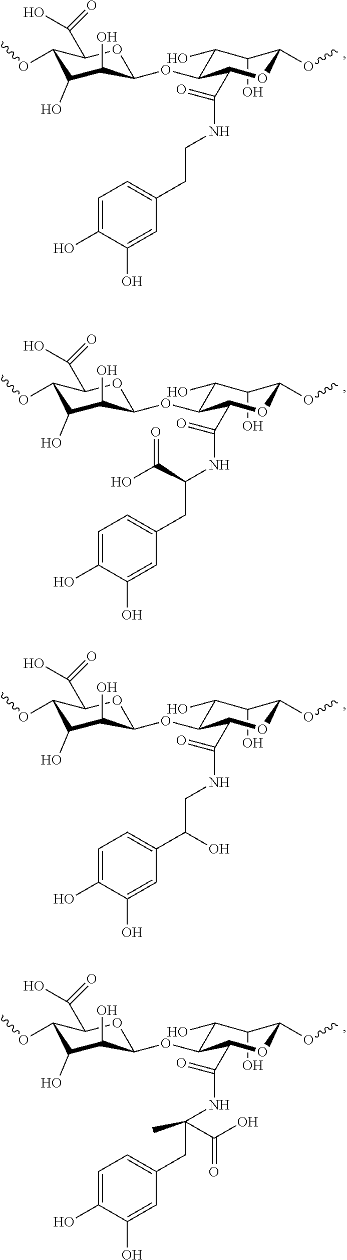

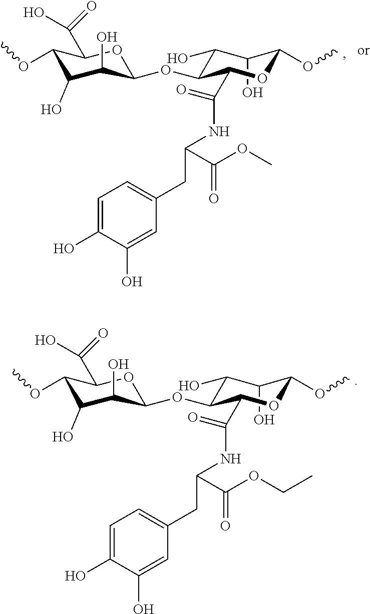

177. The solid dosage form of claim 176, wherein the monomer comprises:

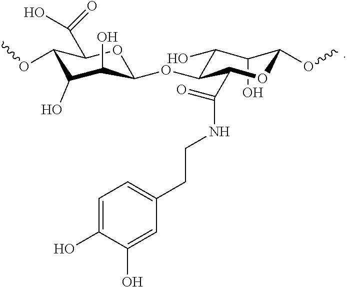

178. A method of treating or preventing a disease or disorder, the method comprising administering an effective amount of the solid dosage form of claim 166 to a subject in need thereof, wherein the disease or disorder is transplant rejection, an eye disease, a joint disease, an infectious disease, a systemic disease, a digestive disorder, bleeding, an ulcer, a bowel obstruction, a parasitic infection, a proliferative disease, a neurological disease, a metabolic disorder, an inflammatory disease, an autoimmune disease, or a psychiatric disease.

179. The method of claim 178, wherein the disease is a metabolic disorder.

180. The method of claim 179, wherein the metabolic disorder is hyperinsulinemia, diabetes, non-alcoholic fatty live disease, nonalcoholic steatohepatitis, or obesity.

181. The method of claim 178, wherein the disease is an infectious disease or a neurological disease.

182. The method of claim 181, wherein the infectious disease is a bacterial infection.

183. The method of claim 181, wherein the neurological disease is Parkinson's Disease.

184. A method of delivering an active pharmaceutical agent to a subject, the method comprising administering to the subject the solid dosage form of claim 166.

185. A method of reducing the dosing frequency of an active pharmaceutical agent, the method comprising administering to a subject the solid dosage form of claim 166.

186. A method of enabling sustained release of an active pharmaceutical agent in a subject, the method comprising administering to the subject the solid dosage form of claim 166.

187. A method of increasing the half-life of an active pharmaceutical agent in a subject, the method comprising administering to the subject the solid dosage form of claim 166.

188. A method of increasing residence time of an active pharmaceutical agent in a subject, the method comprising administering to the subject the solid dosage form of claim 166.

189. A kit comprising:
a solid dosage form of claim 166, and
instructions for administering the solid dosage form to a subject.
